SEQUENCE LISTING (25 sequences)

SEQ ID NO: 1 - protein, 5 aa, Artificial Sequence
  Synthetic
  REPEAT (1)..(5): repeat 10 to 100 times
  Xaa (4)..(4): any amino acid except proline
VPGXG

SEQ ID NO: 2 - protein, 5 aa, Artificial Sequence
  Synthetic
  REPEAT (1)..(5): repeat 80 times
GVGVP

SEQ ID NO: 3 - protein, 5 aa, Artificial Sequence
  Synthetic
  REPEAT (1)..(5): repeat 80 times
  Xaa (2)..(2): Val or Ala at a ratio of 41
GXGVP

SEQ ID NO: 4 - protein, 5 aa, Artificial Sequence
  Synthetic
  REPEAT (1)..(5): repeat 80 times
  Xaa (2)..(2): Val or Ala at a ratio of 11
GXGVP

SEQ ID NO: 5 - protein, 5 aa, Artificial Sequence
  Synthetic
  REPEAT (1)..(5): repeat 80 times
  Xaa (2)..(2): Val or Ala at a ratio of 14
GXGVP

SEQ ID NO: 6 - protein, 9 aa, Artificial Sequence
  Synthetic
  REPEAT (1)..(5): repeat 15 times
  REPEAT (1)..(9): repeat 4 times
  REPEAT (8)..(8): repeat 25 times
GVGVPGDAK

SEQ ID NO: 7 - protein, 9 aa, Artificial Sequence
  Synthetic
  REPEAT (1)..(5): repeat 35 times
  REPEAT (1)..(9): repeat 2 times
  REPEAT (8)..(8): repeat 25 times
GVGVPGDAK

SEQ ID NO: 8 - protein, 9 aa, Artificial Sequence
  Synthetic
  REPEAT (1)..(5): repeat 15 times
  REPEAT (1)..(9): repeat 4 times
  Xaa (2)..(2): Val or Ala at a ratio of 41
  REPEAT (8)..(8): repeat 25 times
GXGVPGDAK

SEQ ID NO: 9 - protein, 9 aa, Artificial Sequence
  Synthetic
  REPEAT (1)..(5): repeat 15 times
  REPEAT (1)..(9): repeat 4 times
  Xaa (2)..(2): Val or Ala at a ratio of 11
  REPEAT (8)..(8): repeat 25 times
GXGVPGDAK

SEQ ID NO: 10 - protein, 9 aa, Artificial Sequence
  Synthetic
  REPEAT (1)..(5): repeat 15 times
  REPEAT (1)..(9): repeat 4 times
  Xaa (2)..(2): Val or Ala at a ratio of 14
  REPEAT (8)..(8): repeat 25 times
GXGVPGDAK

SEQ ID NO: 11 - protein, 9 aa, Artificial Sequence
  Synthetic
  REPEAT (1)..(5): repeat 35 times
  REPEAT (1)..(9): repeat 2 times
  Xaa (2)..(2): Val or Ala at a ratio of 14
  REPEAT (8)..(8): repeat 25 times
GXGVPGDAK

SEQ ID NO: 12 - protein, 5 aa, Artificial Sequence
  Synthetic
  REPEAT (1)..(5): repeat 1 to 200 times
  Xaa (4)..(4): any amino acid except proline
VPGXG

SEQ ID NO: 13 - protein, 3 aa, Artificial Sequence
  Synthetic
  Xaa (1)..(1): Lys, Arg, Asp, or Glu
  REPEAT (1)..(1): repeat 0 to 2 times
  REPEAT (1)..(3): repeat 1 to 50 times
  REPEAT (2)..(2): repeat 1 to 50 times
  Xaa (3)..(3): Lys, Arg, Asp, or Glu
  REPEAT (3)..(3): repeat 0 to 2 times
XAX

SEQ ID NO: 14 - protein, 3 aa, Artificial Sequence
  Synthetic
  Xaa (1)..(1): Lys, Arg, Asp, or Glu
  REPEAT (1)..(2): repeat 1 to 50 times
  REPEAT (1)..(3): repeat 1 to 50 times
  REPEAT (2)..(2): repeat 1 to 5 times
  Xaa (3)..(3): Lys, Arg, Asp, or Glu
  REPEAT (3)..(3): repeat 0 to 2 times
XAX

SEQ ID NO: 15 - protein, 25 aa, Artificial Sequence
  Synthetic
AAAAAAAAAAAAAAAAAAAAAAAAA

SEQ ID NO: 16 - protein, 3 aa, Artificial Sequence
  Synthetic
  REPEAT (2)..(2): repeat 25 times
KAK

SEQ ID NO: 17 - protein, 6 aa, Artificial Sequence
  Synthetic
  REPEAT (1)..(5): repeat 5 times
KAAAAK

SEQ ID NO: 18 - protein, 4 aa, Artificial Sequence
  Synthetic
  REPEAT (3)..(3): repeat 25 times
GDAK

SEQ ID NO: 19 - protein, 15 aa, Artificial Sequence
  Synthetic
  REPEAT (1)..(5): repeat 10 times
  REPEAT (1)..(15): repeat 4 times
  Xaa (7)..(7): para-azidophenylalanine
  REPEAT (11)..(15): repeat 10 times
GVGVPGXGVPGVGVP

SEQ ID NO: 20 - protein, 19 aa, Artificial Sequence
  Synthetic
  REPEAT (1)..(5): repeat 5 times
  REPEAT (1)..(19): repeat 4 times
  Xaa (7)..(7): para-azidophenylalanine
  REPEAT (11)..(15): repeat 10 times
  REPEAT (18)..(18): repeat 25 times
GVGVPGXGVPGVGVPGDAK

SEQ ID NO: 21 - protein, 19 aa, Artificial Sequence
  Synthetic
  REPEAT (1)..(5): repeat 5 times
  REPEAT (1)..(19): repeat 4 times
  Xaa (2)..(2): Val or Ala at a ratio of 14
  Xaa (7)..(7): para-azidophenylalanine
  REPEAT (11)..(15): repeat 10 times
  Xaa (12)..(12): Val or Ala at a ratio of 14
  REPEAT (18)..(18): repeat 25 times
GXGVPGXGVPGXGVPGDAK

SEQ ID NO: 22 - protein, 29 aa, Artificial Sequence
  Synthetic
  REPEAT (1)..(5): repeat 10 times
  REPEAT (1)..(29): repeat 2 times
  Xaa (2)..(2): Val or Ala at a ratio of 14
  Xaa (7)..(7): para-azidophenylalanine
  REPEAT (11)..(15): repeat 15 times
  Xaa (12)..(12): Val or Ala at a Ratio of 14
  Xaa (17)..(17): para-azidophenylalanine
  REPEAT (21)..(25): repeat 10 times
  Xaa (22)..(22): Val or Ala at a ratio of 14
  REPEAT (28)..(28): repeat 25 times
GXGVPGXGVPGXGVPGXGVPGXGVPGDAK

SEQ ID NO: 23 - protein, 10 aa, Artificial Sequence
  Synthetic
  REPEAT (3)..(7): repeat 80 times
MGVPGVGGWP

SEQ ID NO: 24 - protein, 13 aa, Artificial Sequence
  Synthetic
  REPEAT (3)..(7): repeat 35 times
  REPEAT (3)..(10): repeat 2 times
  REPEAT (9)..(9): repeat 25 times
MGVPGVGDAKGWP

SEQ ID NO: 25 - protein, 13 aa, Artificial Sequence
  Synthetic
  REPEAT (3)..(7): repeat 15 times
  REPEAT (3)..(10): repeat 4 times
  REPEAT (9)..(9): repeat 25 times
MGVPGVGDAKGWP

(19) United States
(12) Patent Application Publication
(10) Pub. No.: US 2023/0105233 A1
(43) Pub. Date: Apr. 6, 2023

(54) COMPLEX STRUCTURES FROM STIMULI-RESPONSIVE PROTEINS

(71) Applicant: Duke University, Durham, NC (US)

(72) Inventors: Ashutosh Chilkoti, Durham, NC (US); Stefan Roberts, Durham, NC (US)

(21) Appl. No.: 17/798,868

(22) PCT Filed: Feb. 12, 2021
(86) PCT No.: PCT/US2021/017809

§ 371 Date: Aug. 10, 2022

Related U.S. Application Data
(60) Provisional application No. 62/975,479, filed on Feb. 12, 2020.

(51) Int. Cl. A61K 9/50

(52) U.S. Cl. CPC A61K 9/5052; A61K 9/5089

(57) ABSTRACT

Compositions and methods disclosed herein can provide complex protein-based structures that can be used in biomedical applications. An example composition includes an assembly that includes disordered polypeptides and partially ordered polypeptides.

### CROSS-REFERENCE TO RELATED APPLICATIONS

This application claims priority to U.S. Provisional Patent Application No. 62/975,479 filed on Feb. 12, 2020, which is incorporated fully herein by reference.

### STATEMENT REGARDING FEDERALLY SPONSORED RESEARCH

This invention was made with Government support under Federal Grant No. R35GM127042 awarded by the National Institutes of Health and Federal Grant No. NSF DMR-11-21107 awarded by the National Science Foundation. The Federal Government has certain rights to this invention.

### SEQUENCE LISTING

### BACKGROUND

The ability to control the formation of protein microstructures with complex architectures and spatially segregated regions is useful in numerous applications, including materials science and bioengineering.

### SUMMARY

In one aspect, disclosed are compositions that include a disordered polypeptide having a transition temperature (Tₜ) and comprising an amino acid sequence of [VPGX¹G]ₘ (SEQ ID NO: 1), wherein X¹ is any amino acid except proline and m is 10 to 500; and a partially ordered polypeptide (POP) having a transition temperature of heating (T_{t-heating}) and a transition temperature of cooling (T_{t-cooling}), and comprising a plurality of disordered domains, wherein each disordered domain includes an amino acid sequence of [VPGX²G]ₙ (SEQ ID NO:12), wherein X² is any amino acid except proline and n is 1 to 200, and a plurality of structured domains, wherein each structured domain includes a polyalanine domain, the polyalanine domain comprising at least 5 alanine residues and having at least about 50% of the amino acids in an alpha-helical conformation, wherein the disordered polypeptide's Tₜ is at least ±1° C. compared to the POP's T_{t-heating}.

In another aspect, disclosed are compositions that include an assembly of polypeptides, wherein the polypeptides comprise: a disordered polypeptide having a transition temperature (Tₜ) and comprising an amino acid sequence of [VPGX¹G]ₘ (SEQ ID NO: 1), wherein X¹ is any amino acid except proline and m is 10 to 500; and a partially ordered polypeptide (POP) having a transition temperature of heating (T_{t-heating}) and a transition temperature of cooling (T_{t-cooling}), and comprising a plurality of disordered domains, wherein each disordered domain includes an amino acid sequence of [VPGX²G]ₙ (SEQ ID NO:12), wherein X² is any amino acid except proline and n is 1 to 200, and a plurality of structured domains, wherein each structured domain includes a polyalanine domain, the polyalanine domain comprising at least 5 alanine residues and having at least about 50% of the amino acids in an alpha-helical conformation, wherein the disordered polypeptide's Tₜ is at least ±1° C. compared to the POP's T_{t-heating}.

In another aspect, disclosed are particles that include the disclosed composition.

In another aspect, disclosed are cellular scaffolds that include the disclosed composition, and a plurality of cells, a drug molecule, or a combination thereof.

In another aspect, disclosed are methods of making polypeptide-based assemblies. The method includes adding the disclosed composition to a first solvent to provide a mixture; heating the mixture to a first temperature between the Tₜ of the disordered polypeptide and the T_{t-heating} of the POP to provide a first assembly including one of the disordered polypeptide and the POP; and heating the mixture to a second temperature above the Tₜ of the disordered polypeptide and the T_{t-heating} of the POP to provide a second assembly including the other of the disordered polypeptide and the POP, wherein at least a portion of the second assembly contacts at least a portion of the first assembly to provide an assembly

In another aspect, disclosed are methods of treating a disease or disorder in a subject in need thereof. The method includes administering to the subject a therapeutically effective amount of the disclosed composition.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 shows intrinsically disordered protein (IDP) architectures through elastin-like polypeptides (ELP)-POP mixtures. FIG. 1A: sequence and architecture of example ELPs and POPs. Depicted POPs have a disordered ELP backbone with ordered oligoalanine helices embedded at defined intervals. FIG. 1B: cloud point temperatures (T_{cp}) measured by optical turbidity for ELP(V) and POP(V)−25% (200 μM, PBS). Both IDPs have sharp, lower critical solution temperature (LOST) phase behavior, though POPs can exhibit thermal hysteresis with a lower T_{cp}-cooling than T_{cp}-heating. FIG. 1C: single plane confocal microscopy images above the T_{cp}'s of (i) ELP(V) and (ii) POP(V)−25% (200 μM, PBS). While ELPs form liquid-like coacervates above their T_{cp}), POPs form stable, physically crosslinked porous networks. FIG. 1D: schematic of the formation of a (i) ‘fruits-on-a-vine’ and (ii) core-shell architectures that form based on which component transitions at a lower temperature, FIG. 1E: single plane confocal images of (i-ii) mixtures of POP(V)−25% (200 μM)+ELP(V₄A₁) (200 μM) depicting the ‘fruits-on-a-vine’ architecture and (iii-iv) mixtures of ELP(V) (500 μM)+POP(V₁A₄)−25% (100 μM) depicting the core-shell architecture. Panels ii and iv are higher magnifications of images i and iii, respectively. Both mixtures were imaged after heating from 4° C. to 35° C.—above the T_{cp} of both components—in PBS.

FIG. 2 shows microdroplet architectures. FIG. 2A: depiction of a T-junction microfluidic device that can generate microparticles. FIG. 2B: image analysis of droplets (n=125 droplets) reveals a high degree of monodispersity. FIG. 2C: partial phase diagram for POP(V)−25% illustrating the different discrete states possible during a heating and cooling cycle. FIG. 2D: fluorescence images of POP(V)−25% (500 μM) microdroplets during a heat-cool cycle through the states shown in C. The metastable hysteretic range can prevent dissolution of the particles until the solution temperature is lowered below the T_{cp}-cooling. FIG. 2E: confocal images (25 μm stack) of the same particles in the metastable hysteretic state—state 3. FIG. 2F: partial phase diagram for mixtures of POP(V)−25% (200 μM)+1ELP(V₄A₁) (200 μM) depicting different discrete states possible during a heat-cool cycle of this system in which the POP aggregates at a temperature below the ELP. FIG. 2G: fluorescent images of each state of the cycle demonstrating the formation of a fruits-on-a-vine architecture in state 3. FIG. 2H: confocal images of state 3 depicting the ELP “fruits”. FIG. 21: partial phase diagram of ELP(V) (500 μM)+POP(V₁A₄)−25% (100 μM) depicting states available during a heat-cool cycle. FIG. 2J: fluorescent images of each state of the cycle. Due to the hysteretic nature of the POP and its lower T_{cp}-cooling than the T_{cp} of the ELP, the ELP dissolves first upon cooling, diffusing out and leaving a network of hollow POP shells. FIG. 2K: confocal images of core-shell networks formed in state 3.

FIG. 3 shows controlling hollow shell architecture. FIG. 3A: size distribution of xPOP shells formed after heating a mixture of ELP(V) (1 mM)+xPOP(V₁A₄)−12.5% (100 μM) at different constant rates (10-90% box and whiskers plot with median central line, *p<0.05 as determined by one-way ANOVA with Tukey's post-hoc test, n=50 shells measured using 3D confocal image stacks for each rate). FIG. 3B: histogram of the size distribution of the xPOP shells shows a broad distribution of shell diameters at ramp rates>1° C., the development of a bimodal distribution at 1° C./min, and emergence of a unimodal size at 0.5° C./min. FIG. 3C: fluorescence microscopy images and corresponding cartoon of hollow xPOP shell architectures that form at different heating rates. The system shifts from a network of interconnected hollow shells to a single hollow protein shell as the heating rate is slowed. FIG. 3D: hollow xPOP protein shell diameter increases with increased ELP concentration (10-90% box and whiskers plot with median central line, *p<0.05 as determined by Students t-test, n=50 droplets for each rate). FIG. 3E: linear regression analysis for a polydisperse mixture of ELP(V) (1 mM)+xPOP(V₁A₄)−12.5% (100 μM) comparing diameter of the water-in-oil emulsion droplets with .he diameter of the xPOP shells contained within the droplets (n=280 droplets). FIG. 3F: typical fluorescence images illustrating the linear correlation between droplet diameter and xPOP shell diameter and the ˜0.5 scaling pre-factor that relates droplet diameter to xPOP shell.

FIG. 4 shows extraction into an aqueous environment. FIG. 4A: schematic of a process of extraction from water-in-oil to an all aqueous (buffered saline) environment. FIG. 4B: microscopy images of (i) unextracted (500 μM) and (ii) extracted xPOP(V)−25% porous microparticles and (iii) unextracted and (iv) extracted xPOP shells formed from mixtures of ELP(V) (1 mM)+xPOP (V₁A₄)−12.5% (100 μM) mixtures. The inset in iv demonstrates that hollow shell networks created with faster heating rates can also be extracted. FIG. 4C: scanning electron microscopy (SEM) of a xPOP(V)−25% microparticle showing the interconnected coacervate architecture that comprises the networked particle. FIG. 4D: cryo-SEM images of (i) the outer surface and (ii) a fractured xPOP (V₁A₄)−12.5% shell. The walls of each shell range from approximately 200 to 400 nm in thickness and include tightly packed nano-coacervates of xPOP.

FIG. 5 shows tuning ELP and POP T_{cp}'s. FIG. 5A: example polymer sequences and notation. ELPs and POPs with different ratios of valine (V) and alanine (A) were used to tune polymer transition temperatures (T_{cp}). FIG. 5B: polymer T_{cp} as a function of alanine content (200 μM, PBS) demonstrates the rise in critical temperature with alanine content and the slight depression in T_{cp} caused by the inclusion of oligoalanines in the POP sequences. FIG. 5C: optical density plots (200 μM, PBS) demonstrate the differences in T_{cp-heating} and T_{cp-cooling} caused by changing the mole fraction of helicity within POPs. Increasing helicity can reduce T_{cp} and increase the range of the metastable hysteretic state. FIG. 5D: partial phase diagram for a mixture of ELP(V₁A₄)+POP(V)−25% illustrates the different discrete states available at different temperatures. If heating from 4° C. POP will transition first followed by ELP, and cooling will result in dissolution of ELP first. ‘Either’ indicates that the protein is soluble upon heating from a lower temperature, but remains aggregated upon cooling from a higher temperature. FIG. 5E: partial phase diagram for a mixture of ELP(V)+POP(V₁A₄)−12.5% illustrates the opposing system where heating from 4° C., will cause the ELP to coacervate first, followed by the POP. Because of the metastable hysteretic range, however, ELP will also dissolve first upon cooling.

FIG. 6 shows control of ELP globule size in bulk mixtures. FIG. 6A: confocal image analysis of ELP(V₄A₁) coacervate diameters when heated in PBS with POP(V)−25% (500 μM) at different concentrations reveals that ELP coacervate volume is correlated with ELP concentration. Mixtures were prepared 3 separate times, and an n=150 of ELP “fruit” volumes were collected for each image (data represents mean±sem) FIG. 6B: single plane images of ELP (blue) coacervates within a POP (green) networks show the change in size with concentration. FIG. 6C and FIG. 6D: because ELP aggregates after POP in this mixture, they can be cooled and reheated without change to the POP network. The average coacervate size after reheating is statistically similar (10-90% box and whiskers with median central line bounded by 25 and 75% quartiles, ns with p>0.5 as determined by two-tailed t-test, n=220 ELP ‘fruits’).

FIG. 7 shows tunable sustained release of ELP from stable POP scaffolds. FIG. 7A: fluorescence molecular tomography (FMT) analysis of ELP(V₄A₁) co-injected along with POP(V)−25% subcutaneously in mice demonstrates the sustained-release of ELP from stable POP depots. Comparison between groups (n=5 mice, data represents mean±sem) reveals that the duration of ELP retention is correlated with initial ELP concentration. FIG. 7B: representative FMT images show differences in ELP retention over time. Arrow notes POP depot which has remained at the site of injection despite elution of the ELP.

FIG. 8 shows POP particle shrinking and swelling. FIG. 8A: POP(V)−25% (500 μM) was heated at 5° C./min to 50° C. Using the diameter at 50° C. as a baseline, the swelling and shrinking was measured after cycling between 20° C. and 50° C. at 10° C./min (*p<0.05 as determined by repeated measures ANOVA with Tukey post hoc comparisons, n=5 particles, bar charts represent mean±sem). FIG. 8B: videos of the particles were taken and size was analyzed over the course of the experiment (n=5 particles, data represents mean±sem). The swelling is non-linear, approaching a minimum value at 50° C. FIG. 8C: representative fluorescent microscopy images of the network show the difference in size at the minimum and maximum temperature. FIG. 8D and FIG. 8E: similar analysis was done with hollow POP shells after formation over ELP coacervates. Shells also swell at approximately ˜20% (*p<0.01 as determined by repeated measures ANOVA with Tukey post hoc comparisons, n=130 shells, bar charts represent mean±sem).

FIG. 9 shows mixtures of an ELP and a more hydrophobic POP. FIG. 9A: partial phase diagram of a mixture of ELP(V) (blue)+POP(V₁A₁)−25% (green) showing available states during a cycle of heating and cooling. FIG. 9B: fluorescent images of each stage for a mixture of ELP(V) (500 μM)+POP(V₁A₁)−25% (100 μM) show the formation of core-shell structures and hollow-shell structures similar to mixtures with POP(V₁A₄)−25%. FIG. 9C: if compressed prior to aggregation with a coverslip, the core-shell and hollow shell structures are more visible, though they do not interconnect into a network. FIG. 9D and FIG. 9E: the stages of aggregation and dissolution can be seen more clearly with no interconnection between POP shells. Notably, if the system is heated after dissolving out the ELP, ELP will aggregate and form globules which wet to the outside of the hollow shells (state 5).

FIG. 10 shows unnatural amino acids (UAA) for ultraviolet (UV) crosslinking. FIG. 10A: xPOPs were created by encoding para-azidophenylalanine (pAzF) at 1 residue/100 amino acids. FIG. 10B: partial phase diagram for xPOPs shows that they retain both phase behavior and thermal hysteresis with a slightly downshifted T_{cp} due to the increased hydrophobicity of the aromatic UAA. FIG. 10C: depiction of the pAzF chemical changes during exposure of UV light. FIG. 10D: sodium dodecyl sulphate-polyacrylamide gel electrophoresis (SDS-PAGE) analysis of xPOP(V)−25% crosslinking time. Polymer heated and then exposed to UV light for various times was subsequently cooled, centrifuged, and the resultant supernatant analyzed on an SDS-PAGE gel. Soluble polymers not bound in a network remain in the supernatant and the eventual loss of bands (after 10 s of UV exposure) indicates loss of un-networked polymer. FIG. 10E: fluorescent SDS-PAGE gel of xPOP(V)−25% labeled with DBCO-Cy5. pAzF residues react with DBCO-Cy5, while the negative control, which was expressed without the addition of pAzF to the media, is not fluorescently labeled via click chemistry. FIG. 10F: void volume (determined through analysis of reconstructed confocal images (n=3 unique networks, *p<0.05 as determined by one-way ANOVA with Tukey post hoc comparisons, bar charts represent mean±sem) of xPOP(V)−25% remains unchanged after UV crosslinking. FIG. 10G: confocal images (single plane with 5 μm stack in subset image) of uncrosslinked and UV crosslinked xPOP(V)−25% show no observable differences in architecture.

FIG. 11 shows UV exposure for xPOP microparticles. FIG. 11A: xPOP(V)−25% (500 μM) particles crosslinked after aggregation and exposure to UV light do not solubilize upon cooling, FIG. 11B: UV exposure prior to aggregation does not affect particle formation or reversibility.

FIG. 12 shows an xPOP mixture with a hydrophilic ELP. FIG. 12A: partial phase diagram of a mixture of xPOP(V)−25% ELP(V₄A₁) showing the different states of coacervation achieved when progressing through a cycle of heating->crosslinking->cooling->heating->cooling. FIG. 12B: a single plane confocal image of state 4 of the initial ‘fruits-on-a-vine’ architecture. FIG. 12C: fluorescent images of each state demonstrating the formation, dissolution, and re-formation of the ELP ‘fruits’ (blue) on a crosslinked xPOP particle scaffold (green). FIG. 12D: comparison of ELP ‘fruits’ formed in bulk and on microparticles formed from a mixture of xPOP(V)−25% (200 μM)+ELP(V₄A₁) (200 μM), n=300 ELP ‘fruits’. FIG. 12E: comparison of ELP ‘fruits’ formed on microparticles during initial heating and after dissolution and reheating; n=225 ELP ‘fruits’. For d,e: 10-90% box and whiskers with median central line bounded by 25 and 75% quartiles, *p<0.01 as determined by two-tailed t-test.

FIG. 13 shows 12.5% POP core-shell networks. FIG. 13A: fluorescent images of core-shell structures created with ELP(V) (500 μM, blue)+POP(V₁A₄)−12.5% (100 μM, green). The resultant structures are similar to those formed with 25% POPs though the structures are slightly larger due to the more hydrophilic POP allowing greater time for ELP coacervation (heating rate 1° C./min for left/middle). FIG. 13B: confocal images of the same sample appear different as there is no temperature ramp regulation.

FIG. 14 shows ELP coacervation within xPOP shells. A single plane confocal image taken of a mixture of ELP(V) (1 mM, blue)+xPOP(V₁A₄)−12.5% (100 μM, green) after heating to form a core-shell structure, UV crosslinking to stabilize the shell, cooling to dissolve out the ELP, and finally reheating to re-coacervate the ELP. ELP does not refill the structure, though ELP left inside after diffusion will coacervate and collect on the inside of the xPOP shell.

FIG. 15 shows confocal reconstructions of hollow protein architectures. Mixtures of ELP(V)+xPOP(V₁A₄)−12.5% (100 μM, green) were heated at different rates to produce different final architectures. FIG. 15A: single plane confocal images help elucidate the change in architecture as a function of heating rate depicted in FIG. 3. 20 μm thick confocal stacks of resultant FIG. 15B core-shell networks and FIG. 15C single protein shells reveal their 3D architecture. Small protein aggregates outside of the structures can arise due to minimal surface area for aggregation on ELP, but these are readily removed during extraction. FIG. 15D: linear regression analysis of a polydisperse mixture of ELP(V) (1 mM)+xPOP(V₁A₄)−12.5% (100 μM) heated at 0.5° C./min. The ratio of xPOP shell diameter to the diameter of the emulsion parallels remains constant regardless of emulsion size, providing a means by which to control xPOP shell size (n=280 droplets).

### DETAILED DESCRIPTION

Disclosed herein are complex microarchitectures from stimuli-responsive IDPs, methods of making, and methods of using the same. The formation of intricate microarchitectures typically requires sophisticated fabrication techniques such as flow lithography or multiple-emulsion microfluidics. However, by harnessing the molecular interactions of artificial IDPs, it was found that complex microparticle geometries could be created, including porous particles, core-shell and hollow shell structures, and a unique ‘fruits-on-a-vine’ arrangement, by exploiting the metastable region of the phase diagram of thermally responsive IDPs. In addition, these protein microstructures can be crosslinked and stably extracted to an all-aqueous environment. The complex protein-based structures can be used in a variety of applications, including drug delivery and tissue engineering.

### 1. Definitions

Unless otherwise defined, all technical and scientific terms used herein have the same meaning as commonly understood by one of ordinary skill in the art. In case of conflict, the present document, including definitions, will control. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6,0, 6.1, 6.2, 6.3, 6.1, 6,5, 6.6, 6.7, 6,8, 6.9, and 7.0 are explicitly contemplated.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “amino acid,” as used herein, refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

The terms “effective amount” or “therapeutically effective amount,” as used herein, refer to a sufficient amount of a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses can be the amount of the composition as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration, the type or extent of therapy used, ongoing disease process and type of treatment desired (e.g., aggressive vs. conventional treatment).

The term “expression vector,” as used herein, refers to a plasmid, a virus or another medium, known in the art, into which a nucleic acid sequence for encoding a desired protein can be inserted or introduced.

The term “host cell,” as used herein, refers to a cell that is susceptible to transformation, transfection, transduction, conjugation, and the like with a nucleic acid construct or expression vector. Host cells can be derived from plants, bacteria, yeast, fungi, insects, animals, etc. In some embodiments, the host cell includes Escherichia coli.

The term “polynucleotide,” as used herein, can be single stranded or double stranded, or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

The term “polypeptide,” as used herein, is a linked sequence of two or more amino acids linked by peptide bonds. The polypeptide can be natural, synthetic, or a modification or combination of natural and synthetic. Polypeptides can include proteins such as binding proteins, receptors, and antibodies. The terms “polypeptide” and “protein” are used interchangeably herein. “Primary structure” refers to the amino acid sequence of a particular polypeptide. “Secondary structure” refers to locally ordered, three dimensional structures within a polypeptide. Secondary structure can include alpha helices, beta sheets, and/or others known within the art. These structures are commonly known as domains. “Domains” are portions of a polypeptide that form a compact unit of the polypeptide and are typically 15 to 350 amino acids long.

The term “recombinant,” when used with reference, e.g., to a cell, or nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed, or not expressed at all.

The terms “small molecule,” or “small molecule drug,” as used herein, refer to any small compound, generally less than 1 kDa, which may regulate or control a biological process. In some embodiments, small molecules bind specific biological macromolecules and act as an effector, altering the activity or function of the target. These compounds can be natural, such as secondary metabolites, or synthetic, such as antiviral drugs. They also may have a beneficial effect against a disease, such as drugs or therapeutics.

The term “subject,” as used herein, includes humans and mammals (e.g., mice, rats. pigs, cats, dogs, and horses). Typical subjects of the present disclosure may include mammals, particularly primates and/or humans. For veterinary applications, suitable subjects may include, for example, livestock such as cattle, sheep, goats, cows, swine, and the like; poultry such as chickens, ducks, geese, turkeys, and the like, as well as domesticated animals particularly pets such as dogs and cats. For diagnostic or research applications, suitable subjects may include mammals, such as rodents (e.g., mice, rats, hamsters), rabbits, primates, and swine such as inbred pigs and the like.

The terms “treatment” or “treating,” as used herein, refer to protection of a subject from a disease, preventing, suppressing, repressing, ameliorating, or completely eliminating the disease. Preventing the disease involves administering a composition of the present disclosure to a subject prior to onset of the disease. Suppressing the disease involves administering a composition of the present disclosure to a subject after induction of the disease but before its clinical appearance. Repressing or ameliorating the disease involves administering a composition of the present disclosure to a subject after clinical appearance of the disease.

### 2. Compositions

Disclosed herein are compositions that can include a disordered polypeptide and a POP. These polypeptides may also be referred herein as IDPs. The compositions can also include assemblies of polypeptides, where the polypeptides can include the disordered polypeptide and the POP, or the assemblies can include the POP without the disordered polypeptide.

The ability of the disordered polypeptides and POPs (and compositions thereof) to provide unique structures is aided by the polypeptides being thermally responsive, where they can phase transition at a temperature or temperatures (e.g., a transition temperature—Tₜ, which may also be referred to as a cloud point temperature—T_{cp}). Phase transition refers to the aggregation or assembly of a polypeptide(s) from one state to another. States may include, for example, soluble polypeptides, gels, and aggregates or assemblies of varying size and dimension. The phase transition may occur sharply at or above the Tₜ. In addition, the phase transition may be reversible, although the temperature of dissolution may be the same or different from the temperature of aggregation.

The disordered polypeptide and the POP may have one or more than one transition temperature. Examples of transition temperatures for the polypeptides include a Tₜ, a transition temperature of heating (T_{t-heating}), and a transition temperature of cooling (T_{t-cooling}). Below the Tₜ, for example, a polypeptide may be highly soluble. Upon heating above the transition temperature, for example, a polypeptide may hydrophobically collapse and aggregate, forming a separate phase. In addition, for example, below the T_{t-heating} a polypeptide may be highly soluble. Upon heating above the T_{t-heating}, for example, a polypeptide may hydrophobically collapse and aggregate, forming a separate phase. Then upon cooling below the T_{t-cooling}, for example, the aggregate or assembly may dissolve back into solution. Polypeptides having more than one transition temperature (e.g., having a T_{t-heating} and a T_{t-cooling}) may have each transition temperature correspond to a different individual temperature. This can result in there being a gap between the T_{t-heating} and the T_{t-cooling} (e.g., hysteresis) that allows an aggregate or assembly to be present at a temperature below the T_{t-heating} but above the T_{t-cooling}.

### A. Disordered Polypeptides

The disordered polypeptide is a thermally responsive polypeptide that can have minimal secondary structure as observed by circular dichroism (CD). Secondary structure may also be observed by other techniques known within the art. The disordered polypeptide has phase transition behavior, which allows it to be thermally responsive. The disordered polypeptide may also be responsive to other environmental conditions, such as pH or salt. The phase transition of the disordered polypeptide occurs relative to a Tₜ of the disordered polypeptide. The disordered polypeptide may have a Tₜ of about 0° C. to about 100° C. such as about 10° C. to about 70° C., about 15° C. to about 60° C. about 20° C. to about 50° C., or about 20° C. to about 42° C. The Tₜ of the disordered polypeptide can be adjusted by varying the amino acid sequence, by varying the length of the disordered polypeptide, by varying the concentration of the disordered polypeptide, or a combination thereof. For example, the Tₜ of the disordered polypeptide can be adjusted by varying the guest residue (X¹) with different amino acids. Further discussion on modifying the transition temperature can be found in McDaniel et al., A unified model for de novo design of elastin-like polypeptides with tunable inverse transition temperatures, Biomacromolecules, 2013 August 12; 14(8): 2866-2872, which is incorporated by reference herein in its entirety.

The disordered polypeptide may phase transition at various concentrations. For example, the disordered polypeptide may phase transition at a concentration of about 5 μM to about 20 mM, such as about 5 μM to about 15 mM, about 10 μM to about 10 mM, about 10 μM to about 5 mM, about 10 μM to about 1 mM, about 25 μM to about 800 μM, or about 50 μM to about 500 μM. The disordered polypeptide may phase transition at a concentration that is suitable to form a first assembly, a second assembly, or assembly thereof.

Disordered polypeptides may include elastin-like polypeptides (ELPs). In some embodiments, the disordered polypeptide is an ELP. In some embodiments, the disordered polypeptide includes an amino acid sequence of (VPGX¹G)ₘ(SEQ ID NO:1), wherein X¹ is any amino acid except proline and in is 10 to 500. In some embodiments, m is 15 to 300, 20 to 200, 25 to 150, 30 to 125, or 40 to 100. In some embodiments, X¹ is Val, Ala, or a combination of Val and Ala. In some embodiments, X¹ is a combination of Ala and Val in a ratio from 10:1 to 1:10 (Val:Ala), such as from 7:1 to 1:7 (Val:Ala) or from 4:1 to 1:4 (Val:Ala). In some embodiments, X¹ is Val, V:A at a ratio of 4:1, V:A at a ratio of 1:4, or V:A at a ratio of 1:1.

In some embodiments, the disordered polypeptide includes an amino acid sequence of (SEQ ID NO: 2), (SEQ ID NO: 3), (SEQ ID NO: 4), (SEQ ID NO: 5), or a combination thereof. In some embodiments, the disordered polypeptide includes an amino acid sequence of (SEQ ID NO: 2), (SEQ ID NO: 3), (SEQ ID NO: 4), or (SEQ ID NO: 5).

### B. Partially Ordered Polypeptides (POPS)

The POP has phase transition behavior, which allows it to be thermally responsive. The POP may also be responsive to other environmental conditions, such as pH or salt. The phase transition of the POP occurs relative to a transition temperature of heating (T_{t-heating}) and a transition temperature of cooling (T_{t-cooling}) of the POP. The POP may have a T_{t-heating} and a T_{t-cooling} that are both individually about 0° C. to about 100° C., such as about 10° C. to about 70° C., about 15° C. to about 60° C., about 20° C. to about 50° C., or about 20° C. to about 42° C. The T_{t-heating} and the T_{t-cooling} of the POP can be adjusted by varying the amino acid sequence of the POP (e.g., by varying the amount of the structured domain and/or the X² guest residue), by varying the length of the POP (or domains thereof), or a combination thereof.

The T_{t-heating} and the T_{t-cooling} correspond to two different temperatures. The difference between the two can be referred to as a temperature gap or hysteresis. The POP may have a hysteresis of about 5° C. to about 70° C., such as about 5° C. to about 60° C., or about 10° C. to about 50° C. The POP may have a T_{t-heating} that is at least ±5° C. compared to the T_{t-cooling}, at least ±6° C. compared to the T_{t-cooling}, at least ±7° C. compared to the T_{t-cooling}, at least ±8° C. compared to the T_{t-cooling}, at least ±9° C. compared to the T_{t-cooling}, at least ±10° C. compared to the T_{t-cooling}, at least ±15° C. compared to the T_{t-cooling}, or at least ±20° C. compared to the T_{t-cooling}.

The T_{t-heating}, may be higher than the T_{t-cooling}. For example, the T_{t-heating} can be at least +5° C. compared to the T_{t-cooling}, at least +6° C. compared to the T_{t-cooling}, at least +7° C. compared to the T_{t-cooling}, at least +8° C. compared to the T_{t-cooling}, at least +9° C. compared to the T_{t-cooling}, at least +10° C. compared to the T_{t-cooling}, at least +15° C. compared to the T_{t-cooling}, or at least +20° C. compared to the T_{t-cooling}. In some embodiments, the T_{t-heating} is about 5° C. to about 50° C. higher than the T_{t-cooling}, such as about 5° C. to about 40° C. higher than the T_{t-cooling} or about 5° C. to about 20° C. higher than the T_{t-cooling}.

The POP may phase transition at various concentrations. For example, the POP may phase transition at a concentration of about 5 to about 20 mM, such as about 5 to about 15 mM, about 10 μM to about 10 mM, about 10 μM to about 5 mM, about 10 μM to about 1 mM, about 25 to about 800 μM, or about 50 μM to about 500 μM. The POP may phase transition at a concentration that is suitable to form a first assembly, a second assembly, or assembly thereof.

The POP includes a plurality of disordered domains and a plurality of structured domains. The disordered domains and the structured domains of the POP can be arranged in any number of possible ways. In some embodiments, at least one structured domain is positioned between a plurality of disordered domains of the POP. In some embodiments, one or more disordered domains are positioned between at least two adjacent structured domains of the POP. In some embodiments, the POP includes a plurality of structured domains repeated in tandem and a plurality of disordered domains repeated in tandem. In some embodiments, the POP is arranged as [disordered domain]_{q}-[structured domain]ᵣ-[disordered domain]ₛ-[structured domain]ₜ, wherein q, r, s, and t are independently 0 to 100. In some embodiments, the POP is arranged as [disordered domain]_{q}-[structured domain]ᵣ, wherein q and r are independently 1 to 100. In some embodiments, the POP is arranged as {[disordered domain]_{q}-[structured domain]ᵣ}_{w}, wherein q, r, and w are independently 1 to 100. In some embodiments, q is 1 to 80, 2 to 70. 3 to 60, 5 to 50, or 5 to 40. In some embodiments, r is 1 to 50, 1 to 20, 1 to 10, or 1 to 6. In some embodiments, s is 1 to 80, 2 to 70, 3 to 60, 5 to 50, or 5 to 40. In some embodiments, t is 1 to 50, 1 to 20, 1 to 10, or 1 to 6. In some embodiments, w is 1 to 50, 1 to 20, 2 to 10, or 2 to 8. In some embodiments, the structured domain (or plurality thereof) is positioned C-terminal to the disordered domain (or plurality thereof). In some embodiments, the structured domain (or plurality thereof) is positioned N-terminal to the disordered domain (or plurality thereof).

In some embodiments, the POP includes an amino acid sequence of (SEQ ID NO: 6), (SEQ ID NO: 7). (SEQ ID NO: 8), (SEQ ID NO: 9), (SEQ ID NO: 10), (SEQ ID NO: 11), or a combination thereof. In some embodiments, the POP includes an amino acid sequence of (SEQ ID NO: 6), (SEQ ID NO: 7), (SEQ ID NO: 8), (SEQ ID NO: 9), (SEQ ID NO: 10), or (SEQ ID NO: 11).

### i. Disordered Domains

The disordered domain may have minimal or no secondary structure as observed by CD, while also having phase transition behavior. Disordered domains may include, for example, elastin-like polypeptide (ELP) domains. In some embodiments, the disordered domain is an ELP domain. In some embodiments, the disordered domain includes (VPGX²G)ₙ(SEQ ID NO: 12), wherein X² is any amino acid except proline and n is 1 to 200. In some embodiments, n is 2 to 100, 3 to 75, 5 to 60, 5 to 50, or 10 to 50. In some embodiments, X² is Val, Ala, or a combination of Val and Ala. In some embodiments, X² is a combination of Ala and Val in a ratio from 10:1 to 1:10 (Val:Ala), such as from 7:1 to 1:7 (Val:Ala) or from 4:1 to 1:4 (Val:Ala). In some embodiments, X² is Val, V:A at a ratio of 4:1, V:A at a ratio of 1:4, or V:A at a ratio of 1:1.

The disordered domain can be included in the POP at varying amounts. In some embodiments, about 20% to about 99%, such as about 25% to about 97%, about 35% to about 95%, or about 50% to about 94% of the POP comprises disordered domains. At least about 2%, 3%, 4%, 5%, 10%, 15%, 20%, 25%, 30%, 35%, 40%, 15%, 50%. 55%, 60%, 65%, 70%, 75%, 80%, 85%, 90%, or 95% of the POP may comprise disordered domains.

### ii. Structured Domains

The structured domain may have secondary structure as observed by CD, such as, for example, an alpha helix, The structured domain may include a polyalanine domain. Each polyalanine domain may include at least 5 alanine residues. Each polyalanine domain may include 5 to 50 alanine residues, such as 6 to 45 alanine residues, 7 to 40 alanine residues, 8 to 35 alanine residues, or 5 to 30 alanine residues. In addition, each polyalanine domain may have at least about 50% of the amino acids in an alpha-helical conformation. In some embodiments, each polyalanine domain has at least 60% of the amino acids in an alpha-helical conformation.

In some embodiments, the polyalanine domain comprises an amino acid sequence of [Bₚ(A)_{q}Zᵣ]ₙ (SEQ ID NO: 13) or [(BAₛ)ₜZᵣ]ₙ (SEQ ID NO: 14), or a combination thereof, wherein B is Lys, Arg, Asp, or Glu; A is Ala; Z is Lys, Arg, Asp, or Glu; n is 1 to 50; p is 0 to 2; q is 1 to 50; r is 0 to 2; s is 1 to 5; and t is 1 to 50.

In some embodiments, the structured domain includes (A)₂₅ (SEQ ID NO: 15), K(A)₂₅K (SEQ ID NO: 16), (KAAAA)₅K (SEQ ID NO: 17), GD(A)₂₅K (SEQ ID NO: 18), or a combination thereof. In some embodiments, the structured domain includes an amino acid sequence of (A)₂₅ (SEQ ID NO: 15), K(A)₂₅K (SEQ ID NO: 16), (KAAAA)₅K (SEQ ID NO: 17), or GD(A)₂₅K (SEQ ID NO: 18). In some embodiments, the structured domain includes GD(A)₂₅K (SEQ ID NO: 18).

The structured domain can be included in the POP at varying amounts. In some embodiments, about 4% to about 75%, such as about 5% to about 70%, about 6% to about 60%, or about 7% to about 50% of the POP comprises structured domains. At least about 2%, 3%, 4%, 5%, 10%, 15%, 20%, 25%, 30%, 35%, 40%, 45%, 50%, 55%, 60%, 65%, 70%, 75%, 80%, 85%, 90%, or 95% of the POP may comprise structured domains.

Further discussion of the POP can be found in U.S. patent application Ser. No. 16/625,899, which is incorporated by reference herein in its entirety.

### C. Crosslinking Moieties

The disordered polypeptide, the POP, or both can include a crosslinking moiety. The crosslinking moiety can be any suitable moiety known within the art that allows the polypeptides to be covalently crosslinked with other polypeptides, within the same polypeptide, or both. Examples of crosslinking moieties include, but are not limited to, unnatural amino acid derivatives (e.g., para-azidophenylalanine), sequences encoded within the polypeptide (e.g., an enzymatic recognition site), and naturally occurring side chains of amino acids within the polypeptide (e.g. using side chain of lysine present within a polypeptide). Accordingly, different crosslinking agents can be used to form the covalent crosslinks. Examples include, but are not limited to, an external stimulus (e.g., UV light), small molecules that can target one or more amino acids (e.g., tetrakis (hydroxylmethyl) phosphonium chloride), and enzymes that can target a specific amino acid(s) or encoded sequence (e.g., lysyl oxidase).

In some embodiments, the disordered polypeptide, the POP, or both may include amino acid derivatives that are not naturally occurring, such as a UV crosslinkable amino acid derivative. The non-native amino acid derivative can be used to introduce covalent crosslinks between different polypeptides and within the same polypeptide. For example, polypeptides that include the UV crosslinkable amino acid derivative can be exposed to UV light, which can result in covalent crosslinks being formed between the amino acid derivative and a side chain of an amino acid of another polypeptide or with a side chain of an amino acid of the same polypeptide (having the amino acid derivative). The UV crosslinkable amino acid derivative may be any amino acid that has been functionalized with an azide group. In some embodiments, the amino acid derivative is para-azidophenylalanine.

The UV crosslinkable amino acid derivative may be included at varying amounts without affecting the disordered polypeptide's or the POP's ability to transition at different temperatures. For example, the UV crosslinkable amino acid derivative may be included within the disordered polypeptide, the POP, or both from about 0.1% to about 20% (of the polypeptide), such as from about 0.5% to about 15% (of the polypeptide) or from about 1% to about 10% (of the polypeptide).

In embodiments that include a crosslinking moiety, the disordered polypeptide, the POP, or both can include an amino acid sequence of (SEQ ID NO: 19), (SEQ ID NO: 20), (SEQ ID NO: 21), (SEQ ID NO: 22), or a combination thereof. In some embodiments, the disordered polypeptide, the POP, or both includes an amino acid sequence of (SEQ ID NO: 19), (SEQ ID NO: 20), (SEQ ID NO: 21), or (SEQ ID NO: 22).

### D. Assemblies

Because the disordered polypeptides and the POPs can have tailored transition temperatures, their thermal responsiveness can be used to form unique protein-based structures. For example, the disordered polypeptide and the POP can have different transition temperature(s), allowing one of them to phase transition before the other. The difference of transition temperatures can be reflected as a difference between the Tₜ of the disordered polypeptide and the T_{t-heating} or the T_{t-cooling} of the POP. For example, the disordered polypeptide's Tₜ can be at least ±1° C. compared to the POP's T_{t-heating}, at least ±2° C. compared to the POP's T_{t-heating}, at least ±3° C. compared to the POP's T_{t-heating}, at least ±4° C. compared to the POP's T_{t-heating}, at least ±5° C. compared to the POP's T_{t-heating}, at least ±10° C. compared to the POP's T_{t-heating}, at least ±15° C. compared to the POP's T_{t-heating}, at least ±20° C. compared to the POP's T_{t-heating}, or at least ±30° C. compared to the POP's T_{t-heating}. The difference between the Tₜ of the disordered polypeptide and the T_{t-heating} of the POP can be about 1° C. to about 100° C., such as about 1° C. to about 75° C., about 1° C. to about 50° C., about 1° C. to about 30° C., about 2° C. to about 80° C., about 3° C. to about 70° C., or about 5° C. to about 100° C. This foregoing description regarding the disordered polypeptide's Tₜ and the POP's T_{t-heating} can also be used to describe the difference between disordered polypeptide's Tₜ and the POP's T_{t-cooling}.

In some embodiments, the disordered polypeptide phase transitions prior to the POP. In other embodiments, the POP phase transitions prior to the disordered polypeptide. The polypeptide that transitions first can provide a structure in which the polypeptide that transitions second can associate with during its phase transition. Accordingly, in some embodiments, the assembly includes a first assembly including one of the disordered polypeptide and the POP (e.g., the first assembly includes the polypeptide that phase transitions first), and a second assembly including the other of the disordered polypeptide and the POP (e.g., the second assembly includes the polypeptide that phase transitions second), wherein at least a portion of the first assembly contacts at least a portion of the second assembly. Furthermore, one of the first assembly and the second assembly can be released from the assembly over time. For example, a first assembly that includes the POP and a second assembly that includes the disordered polypeptide can release the disordered polypeptide from the assembly over time.

As indicated above, the polypeptides of the compositions can self-assemble into the assembly in different phases. In some embodiments, the polypeptides self-assemble into the assembly in two phases relative to the Tₜ of the disordered polypeptide and the T_{t-heating} of the POP. These two phases can include: (1) a first phase at a temperature between the Tₜ of the disordered polypeptide and the T_{t-heating} of the POP, wherein one of the disordered polypeptide and the POP self-assembles into the first assembly; and (2) a second phase at a temperature above the Tₜ of the disordered polypeptide and the T_{t-heating} of the POP, wherein the other of the disordered polypeptide and the POP self-assembles into the second assembly, and wherein at least a portion of the second assembly contacts at least a portion of the first assembly, the combination of the first assembly and second assembly providing the assembly.

The self-assembly of the polypeptides can also include a third phase. The third phase transition can occur subsequently after the second phase transition has occurred. The third phase can include a temperature below the Tₜ of the disordered polypeptide and above the T_{t-cooling} of the POP, wherein the disordered polypeptide dissolves from the assembly. The resultant assembly after the third phase transition can include the POP, but not the disordered polypeptide.

The assembly can be provided in many unique and complex structures. Examples include, but are not limited to, networks with associated particles (also referred to as “fruit-on-a-vine” structure), porous particles, core-shell particles, and hollow particles. In some embodiments, the assembly includes a porous network including the POP, and a plurality of particles contacting the network, each particle including the disordered poly peptide. The particles in contact with the network can each, individually have a diameter of about 0.2 μm to about 200 μm, such as about 0.5 μm to about 100 μm or about 1 μm to about 50 μm. The size of the particles in contact with the network can have their size varied according to the concentration of the disordered polypeptide in the composition.

In some embodiments, the assembly includes a plurality of particles, where each particle includes a core that includes the disordered polypeptide, and a shell that includes the POP—where the shell is positioned on a surface of the core. The particles can each, individually have a diameter of about 0.1 μm to about 200 μm, such as about 0.15 μm to about 100 μm or about 0.2 μm to about 50 μm. The size of the particles can be controlled by the difference in transition temperatures between the disordered polypeptide and the POP. In addition, the shell can be porous and can have physical crosslinks between individual POPs.

In some embodiments, after a third phase transition, the assembly can include (i) a porous network including the POP; or (ii) a plurality of hollow shell particles, each hollow shell particle including the POP. The hollow shell particle can have a shell that can vary in thickness. For example, the shell of the hollow shell particle may have a thickness of about 200 nm to about 400 nm.

In addition, other particulate assemblies can be provided by compositions that include the POP but do not include the disordered polypeptide. As such, these particulate assemblies include the POP, but not the disordered polypeptide. In these embodiments, the composition includes a particle of polypeptides, wherein the polypeptide includes the POP as described above.

As mentioned above, the disordered polypeptide, the POP, or both can include crosslinking moieties. Accordingly, the first assembly, the second assembly, or the assembly can include crosslinks between the polypeptides (e.g., after undergoing a crosslinking reaction).

### E. Drug Molecules

The composition can include a drug molecule. Drug molecules can be advantageous in methods of using the disclosed compositions, such as treating disease, tissue engineering, drug delivery, and the like. Examples of drug molecules include, but are not limited to, a small molecule, a polypeptide, a polynucleotide, a lipid, a carbohydrate, or a combination thereof. The drug molecule can be encapsulated within the first assembly, the second assembly, or the assembly. The drug molecule can be encapsulated within different assemblies by taking advantage of the thermal responsiveness of the disclosed polypeptides.

### 3. Polynucleotides

Further provided are polynucleotides encoding the polypeptides detailed herein. A vector may include the polynucleotide encoding the disordered polypeptide, the POP, or both detailed herein. To obtain expression of a polypeptide, one may subclone the polynucleotide encoding the polypeptide into an expression vector that contains a promoter to direct transcription, a transcription/translation terminator, and if for a nucleic acid encoding a protein, a ribosome binding site for translational initiation. An example of a vector is pet24. Suitable bacterial promoters are well known in the art. Further provided is a host cell transformed or transfected with an expression vector comprising a polynucleotide encoding the disordered polypeptide, the POP, or both as detailed herein. Bacterial expression systems for expressing the protein are available in, e.g., E. coli, Bacillus sp., and Salmonella (Paiva et al., Gene 1983, 22, 229-235; Mosbach et al., Nature 1983, 302, 543-545—both of which are incorporated by reference in their entirety herein). Kits for such expression systems are commercially available. Eukaryotic expression systems for mammalian cells, yeast, and insect cells are well known in the art and are also commercially available. Retroviral expression systems can also be used.

The disordered polypeptide and the POP may be expressed recombinantly in a host cell according to one of skill in the art. The disordered polypeptide and the POP may be purified by any means known to one of skill in the art. For example, the disordered polypeptide and the POP may be purified using chromatography, such as liquid chromatography, size exclusion chromatography, affinity chromatography, or a combination thereof. In some embodiments, the disordered polypeptide and the POP are purified without chromatography. In some embodiments, the disordered polypeptide and the POP are purified using inverse transition cycling.

### 4. Particles Including the Compositions

Also disclosed herein are particles that include the disclosed compositions. As discussed in more detail below, emulsification methods can be used to prepare particles that include the disclosed compositions with and without the associated assemblies. Particles prepared by emulsification can be emulsion droplets. Particles prepared by emulsification can also be a particle of the assembly (and composition thereof)—e.g., when the emulsion droplet including the assembly (and composition thereof) is extracted into an aqueous phase.

The particles including the disclosed compositions can be referred to as microparticles or microparticle sized emulsion droplets. The particles including the disclosed compositions can each, individually have a diameter of about 10 μm to about 500 μm, such as about 10 μm to about 200 μm, about 15 μm to about 150 μm, or about 20 μm to about 100 μm. The size of the particle can be controlled by the concentration of the disordered polypeptide, the POP, or both in the composition. In addition, in embodiments where microfluidics are used to provide the particles including the composition, different microfluidic parameters can be used to vary the size of the particle, such as varying the diameter of the channel of a microfluidic device.

In some embodiments, the particle includes the disclosed composition where the assembly includes a porous network including the POP; and a plurality of particles contacting the network, each particle including the disordered polypeptide. In some embodiments, the particle includes the disclosed composition where the assembly includes a network of network particles, each network particle including a core including the disordered polypeptide; and a shell including the POP, wherein the shell is positioned on a surface of the core.

In some embodiments, the particle includes the disclosed composition where the assembly includes (i) a porous network including the POP; or (ii) a network of hollow shell particles, each hollow shell particle including the POP. These latter embodiments are provided by including a third phase transition, where the disordered polypeptide is no longer part of the assembly.

The particles can include assemblies that have been crosslinked. In embodiments where the assemblies have been crosslinked, the particles thereof can have useful physical properties. For example, the particle can have a Young's modulus of about 1 kPa to about 80 kPa, such as about 5 kPa to about 50 kPa or about 10 kPa to about 30 kPa. This can allow the particles to be extracted into aqueous phases.

The description of the compositions, disordered polypeptides, POPs, and assemblies can also be applied to the particles including the compositions disclosed herein.

### 5. Scaffolds

Further provided herein are scaffolds (also referred to as a cellular scaffold) that include the disclosed compositions. The scaffold can include a plurality of cells, a drug molecule, or a combination thereof. The cells may include a variety of types. In some embodiments, the cells comprise stem cells, bacterial cells, human tissue cells, or a combination thereof. The cells may be encapsulated in the first assembly, the second assembly, or the assembly. Further description on the drug molecule can be found above.

The scaffold may have low immunogenicity or low antigenicity or both. The scaffold may promote at least one of cell growth, cell recruitment, and cell differentiation, or a combination thereof. The scaffold may be suitable for cell transplantation, tissue regeneration, cell culture, and/or cell-based in vitro assays. In addition, the scaffold may promote the formation of vasculature, wound healing, or a combination thereof.

The disclosed compositions can provide a facile system that can allow for one of the disordered polypeptide and the POP to elute from the assembly prior to the other. This property can be useful for, e.g., a drug eluting scaffold. In some embodiments, the scaffold includes a disclosed composition that includes an assembly that comprises a porous network including the POP, and a plurality of particles contacting the network, each particle including the disordered polypeptide.

The description of the compositions, disordered polypeptides. POPs, and assemblies can also be applied to the scaffolds including the compositions disclosed herein.

### 6. Methods of Making Assemblies

Provided herein are methods of making protein-based assemblies having unique structures. The method can include adding the disclosed compositions to a first solvent to provide a mixture. The first solvent can be any solvent that dissolves or partially dissolves the disordered polypeptide, the POP, and the compositions thereof. In some embodiments, the first solvent is an aqueous solvent.

The method can include heating the mixture to a first temperature between the Tₜ of the disordered polypeptide and the T_{t-heating} of the POP to provide a first assembly including one of the disordered polypeptide and the POP. The method can also include heating the mixture to a second temperature above the Tₜ of the disordered polypeptide and the T_{t-heating} of the POP to provide a second assembly including the other of the disordered polypeptide and the POP, wherein at least a portion of the second assembly contacts at least a portion of the first assembly to provide the assembly. In addition, the rate of the heating can be used to control architecture of resultant assemblies. In some embodiments, the first heating step, the second heating step, or both can be done at a rate of about 0.5° C./min to about 20° C./min.

The method can also include cooling the mixture to a third temperature below the Tₜ of the disordered polypeptide and above the T_{t-cooling} of the POP, wherein the disordered polypeptide dissolves from the assembly. This cooling step can be performed after the second heating step. In addition, including this cooling step can allow for assemblies that include the POP, but not the ELP. Example structures provided by the added cooling step include, but are not limited to, a porous network including the POP and a plurality of hollow shell particles, each hollow shell particle including the POP.

The heating and cooling steps of the methods can be repeated a number of times. For example, in methods that include the cooling step, the mixture can then be heated again to provide the second assembly. In addition, in methods that include the cooling step, the mixture can be further cooled below the Tₜ of the disordered polypeptide, the T_{t-heating} of the POP, and the T_{t-cooling} of the POP, wherein the POP dissolves from the assembly. Thus, the mixture can be returned to no assemblies being formed.

The disclosed method can also be implemented via an emulsion process. This includes, but is not limited to, bulk emulsions and microfluidic mediated emulsions. The method can include emulsifying the mixture prior to any of the heating steps. This can include forming emulsion droplets that include the disclosed compositions. Thus, when the emulsion droplets undergo the heating steps, the first assembly, the second assembly, and/or the assembly can be present in an emulsion droplet.

The method can further include exposing the different assemblies to a crosslinking agent, such as UV light. For example, the method can include exposing the first assembly, the second assembly, or a combination thereof (e.g., the assembly) to a crosslinking agent. Exposing the different assemblies to a crosslinking agent can allow for crosslinks to be formed between the polypeptides. This can provide a more robust structure, which can allow for more effective extraction of the assemblies and compositions thereof into a solvent of choice. As such, the method can also include extracting the first assembly, the second assembly, or the assembly into an aqueous phase. This can be useful when the assemblies are formed via an emulsion process. This can allow the provided assemblies and compositions thereof to be extracted from an oil phase into an aqueous phase. Furthermore, the use of a crosslinking agent can be used in methods with or without emulsification.

The description of the compositions, particles including the compositions, disordered polypeptides, POPs, and assemblies can also be applied to the methods of making disclosed herein.

### 7. Methods of Treatment

The present disclosure also provides methods of treating a disease or disorder in a subject in need thereof. The method may include administering to the subject a therapeutically effective amount of the disclosed composition. The description of the compositions, particles including the compositions, disordered polypeptides, POPs, and assemblies can also be applied to the methods of treatment disclosed herein.

### 8. EXAMPLES

### Example 1

### Materials and Methods

### Gene Expression

ELPs and POPs: Single stranded oligonucleotides encoding the polymer genes were purchased from Integrated DNA Technologies (IDT) and cloned into a modified pet-24 vector via recursive directional ligation by plasmid reconstruction into chemically competent Eb5α E. coli to assemble the full-length polymer genes, see Oh, J. K., Drumright, R., Siegwart, D. J. & Matyjaszewski, K. The development of microgels/nanogels for drug delivery applications. Progress in Polymer Science 33, 448-477, which is incorporated by reference herein in its entirety. In brief, A and B populations of each gene fragment were generated by restriction digest with AcuI and BglI and BseRI and BglI, respectively. Ligation of appropriate plasmid fragments from A and B populations following DNA gel purification resulted in the formation of a sing le, concatenated A+B gene fragment inside the modified pet-24 vector.

xPOPs: Following their full-length assembly using the above methods, xPOP genes were further isolated via BseRI and BamHI restriction digest, and the isolated gene was cloned into another modified vector with a pTac promoter and rrnB terminator instead of the T7 promoter and terminator of the original vector. The plasmids were then co-transformed into c321. ΔA E. coli alongside a pEvol tRNA/aaRS vector with two copies of pAcFRS.1.t1 synthetase. The c321.ΔA genome has previously been edited to remove all instances of the amber stop codon, and the tRNA/aaRS pair has been optimized to recognize the amber stop codon and incorporate para-azidophenylalanine, see Costa, S. A. et al. Photo-crosslinkable unnatural amino acids enable facile synthesis of thermoresponsive nano- to microgels of intrinsically disordered polypeptides. Adv Mater 30, (2018) and Amiram, M. et al. Evolution of translation machinery in recoded bacteria enables multi-site incorporation of nonstandard amino acids. Nat Biotechnol 33, 1272-1279, (2015), both of which are incorporated by reference in their entirety herein.

### Biopolymer Synthesis and Characterization

ELP expression: Liquid cell cultures from 25% glycerol stocks were grown overnight (˜46 h) in 25 mL 2xYT starter cultures containing 45 μg/mL kanamycin at 37° C. and 200 rpm. Starter cultures were then transferred to 1 L 2xYT cultures the following morning and grown for ˜8 37° C. at 200 rpm in the presence of 45 μg/mL kanamycin. 1 mM IPTG was then added to induce expression, and cultures were grown for an additional ˜16 h overnight at 37° C. and 200 rpm.

POP expression: POP expression follows a nearly identical protocol as ELPs, however, cultures are grown at 25° C. prior to induction with IPTG and at 16° C. overnight after induction to reduce the formation of truncation products from ribosomal pausing.

xPOP expression: xPOP expression follows a similar protocol with two exceptions. (1) 45 μg/mL kanamycin, 25 μg/mL chloramphenicol, 0.2% arabinose, and 1 mM pAzF are included in all cultures from inoculation, (2) cultures are grown at 34° C., determined to be the optimal temperature for pAzF incorporation.

Purification: For all IDPs, cell pellets were collected via centrifugation at 3500 rpm for 10 min and resuspended in PBS to an appropriate volume (˜25 mL). Samples were sonicated for a total of 3 min to lyse cells, and supernatant was collected following centrifugation at 14000 rpm for 10 min at 4° C. 2 mL/L culture of 10% PEI was added, and the supernatant was collected following centrifugation at 14000 rpm for 10 min at 4° C. Three rounds of inverse transition cycling (ITC), a method that utilizes the thermally responsive properties of the IDPs, were then performed to purify the IDPs. Briefly, samples were heated to 50° C. (supplemented with 2M NaCl in the first cycle) and centrifuged at 14000 rpm and 30° C. for 10 min. Then, pellets were resuspended in PBS and put through another centrifugation step at 14000 rpm and 4° C. for 10 min in each cycle. Purity was determined via SDS-PAGE gel electrophoresis. Samples were then dialyzed into water, lyophilized, and stored at −20° C. All protocols for xPOP expression and purification were completed under low-light conditions to avoid undesirable pAzF crosslinking during synthesis and purification.

### Turbidity

Coacervation behavior was characterized using a Cary 100 UV-Vis spectrophotometer monitoring optical density at 350 nm (or 650 nm for xPOPs as pAzF absorbance interferes at 350). Samples in 1× PBS were heated and cooled at 1° C./min and the temperatures at which the first derivative of the curve was the maximum were defined as the T_{cp}—heating and—cooling.

### Microparticle Synthesis and Extraction

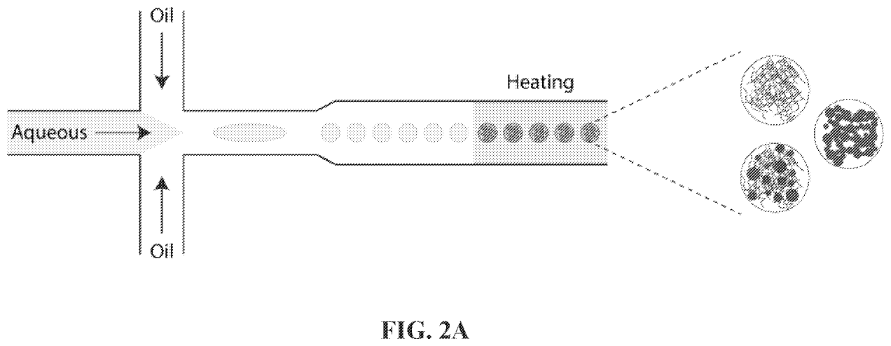

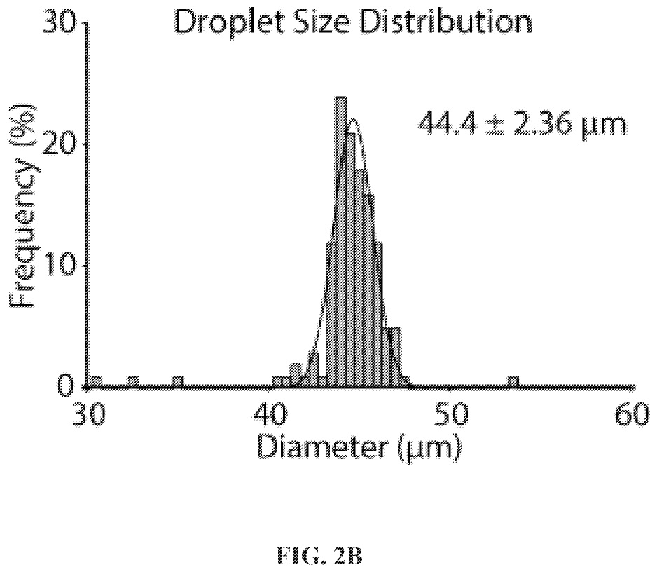

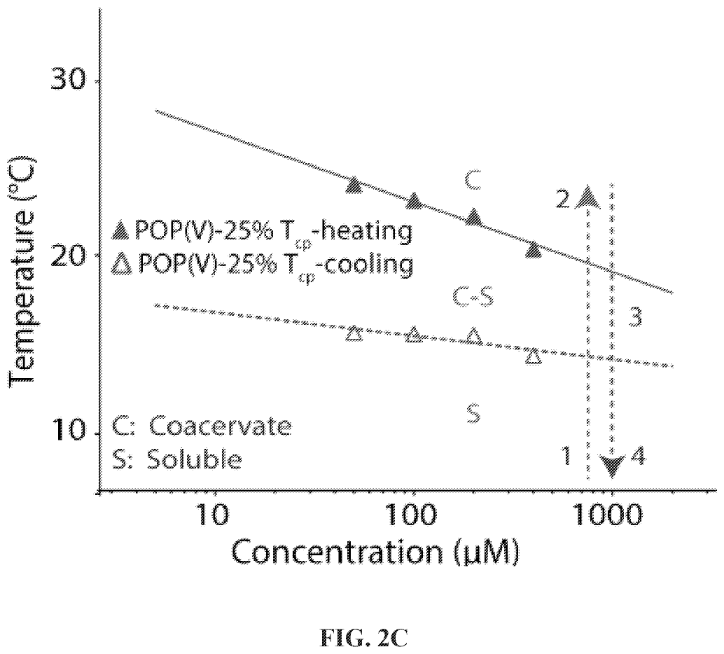

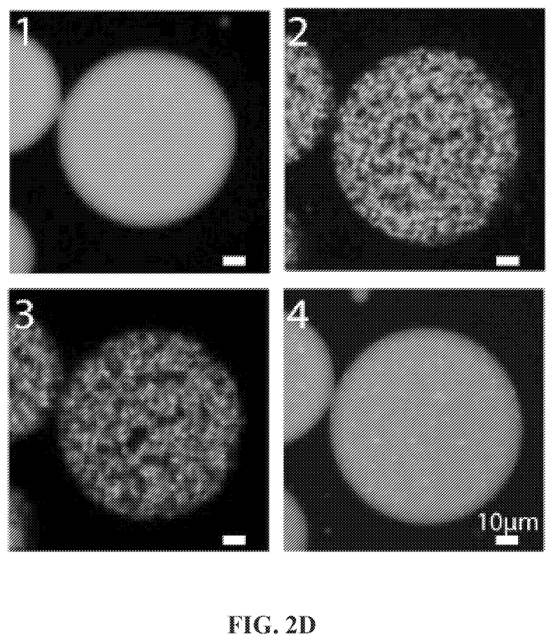

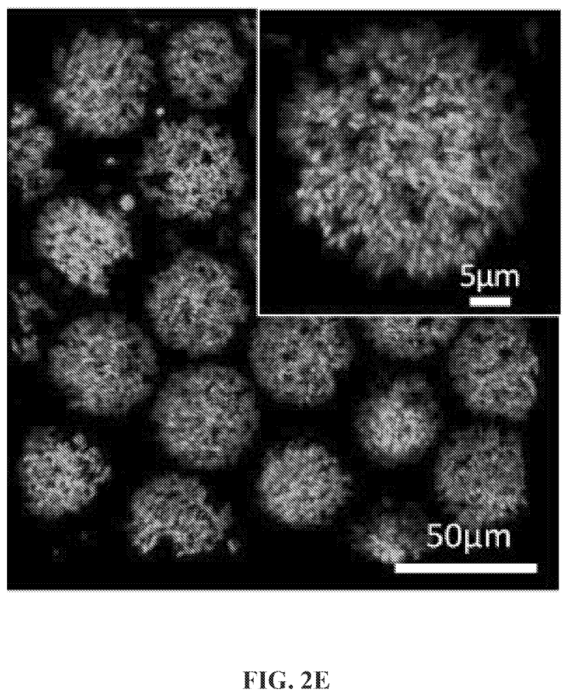

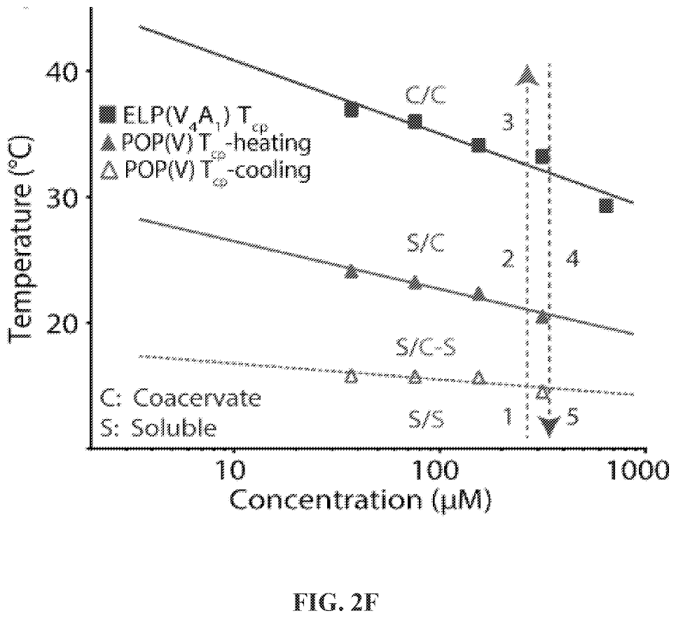

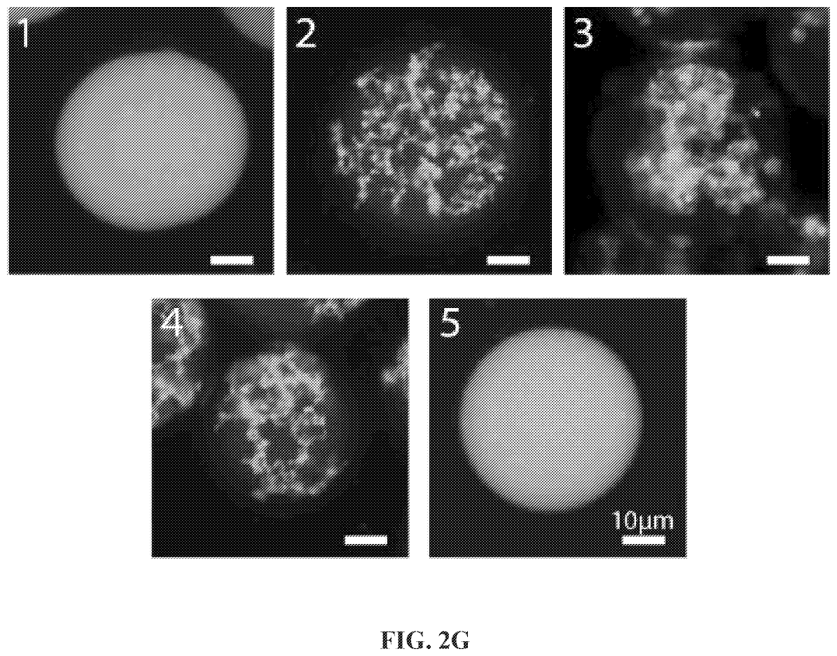

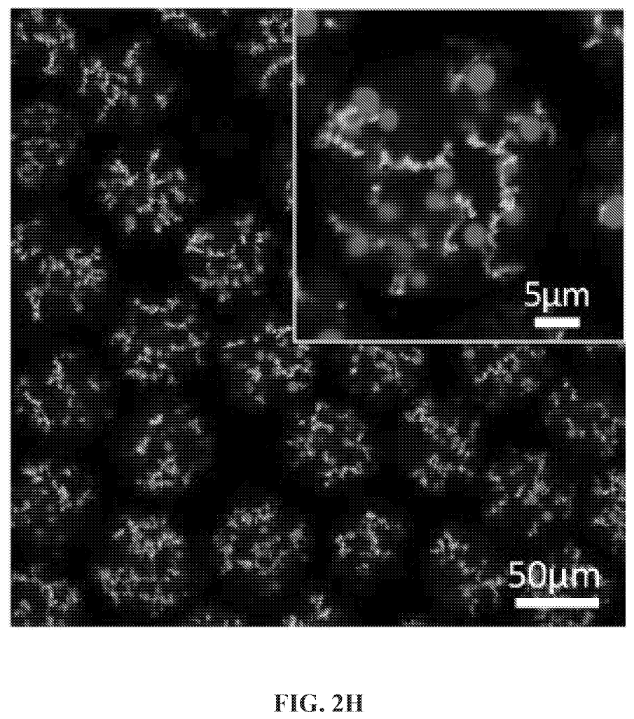

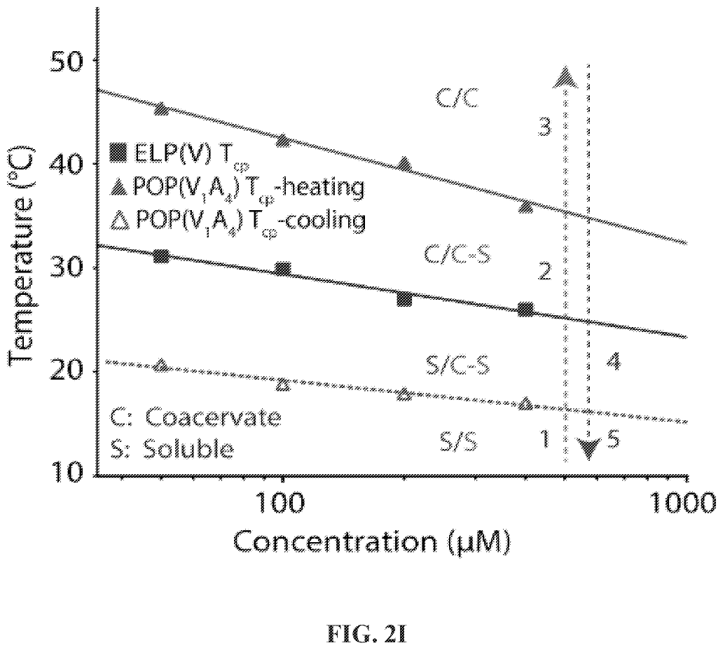

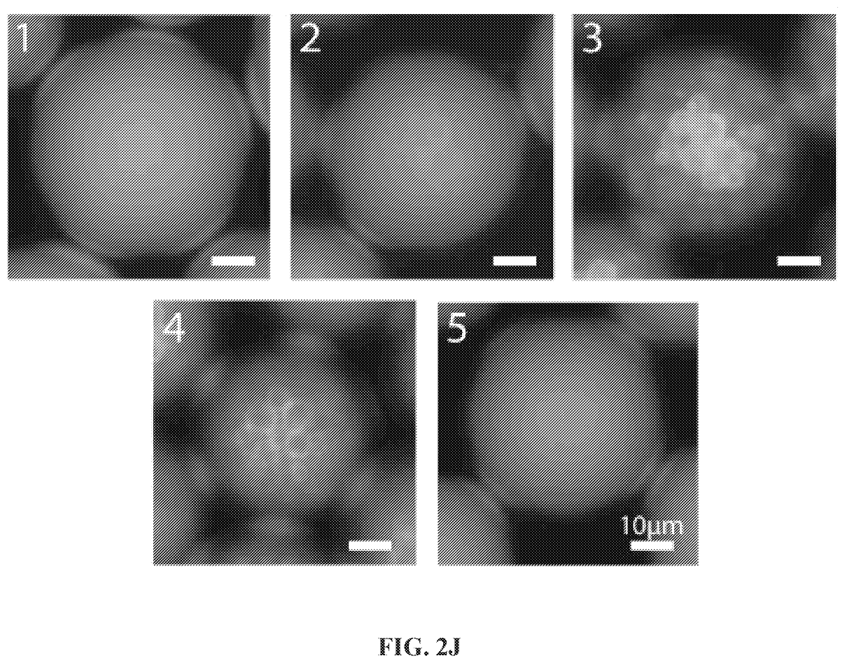

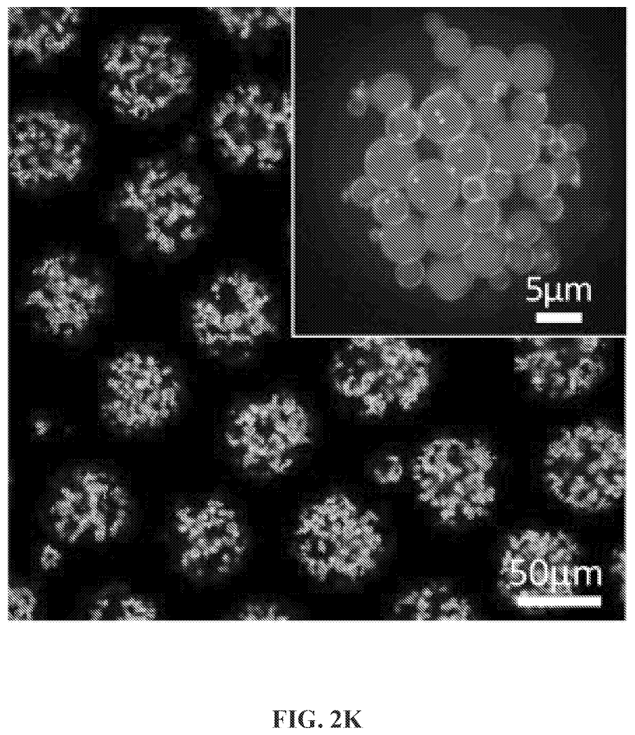

Microparticles were generated in water-in-oil emulsions using droplet microfluidics (FIG. 2A) as described previously in Simon et al., Programming molecular self-assembly of intrinsically disordered proteins containing sequences of low complexity. Nat Chem 9, 509-515, (2017), which is incorporated by reference herein in its entirety. In brief, precision syringe pumps were used to flow an aqueous phase at 50-150 μL/hr (variation in flow rate was used to control size of the microparticles) and an oil phase composed of 75%/5%/20% v/v TEGOSOFT DEC/ABIL EM 90/mineral oil at 250 μL/hr through a polydimethylsiloxane (PDMS) microparticle generator. Microparticles were generated at 4° C. to prevent polymer aggregation during the process. Microemulsions were stable for ˜1 week when stored at 4° C. before loss of monodispersity. Where necessary, an Omincure Series 1000 lamp was used to crosslink samples (30 s, 50% power, 100 W lamp, 312 nm) in emulsion.

To extract microparticles back into an aqueous environment, water-in-oil emulsions were gently mixed 1:10 v/v into isobutanol, and hand-rotated for 30 s. Samples were centrifuged at 1000 rpm at 4° C. for 5 min to pellet microparticles, and the oil phase removed. Microparticles were then twice washed with Ix PBS and centrifuged at 1000 rpm for 5 min. Microparticles were then resuspended in 1× PBS for microscopy.

### Microscopy

The IDPs were fluorescently labeled using either Alexa Fluor 350 or Alexa Fluor 488 NHS Ester with a typical reaction efficiency of ˜50%. Excess dye was removed with dialysis and IDPs were lyophilized for storage. For all experiments, the fluorescently-labeled IDPs were mixed with the unlabeled IDP such that <10% mole fraction of POPs in solution were labelled.

Optical fluorescence microscopy was performed on an upright Zeiss Axio Imager A2 microscope with a Zeiss Incubation System S heating stage and PeCon TempController 2000-2 temperature control unit. Unless otherwise stated, samples were heated and cooled at 5° C./min. Sample drying was problematic at higher temperature with heating rates of 0.5° C./min and 1° C./min. As a result, these samples were heated and cooled in an Arktik thermal cycler and then rapidly (<10 s) transferred to the microscope stage that was pre-heated to the final desired temperature. Confocal microscopy was performed on a Zeiss 710 inverted confocal microscope with an environmental heating chamber. All samples were imaged at either 25° C. or 35° C. after equilibration at the appropriate temperature. All images were processed and analyzed in ImageJ. Where possible, a standardized image processing cascade using thresholding and the ‘analyze particles’ plugin was used to automate size calculations. In rare cases, manual line segments were also drawn.

Scanning electron microscopy was performed on a FEI XL30 scanning electron microscope. Microdroplets extracted into PBS were allowed to dry at room temperature and sputter coated with gold prior to imaging. Cryo-SEM was performed on a JEOL JSM-7600F SEM outfitted with a cryogenic transfer system. Samples were flash frozen in liquid nitrogen slush and transferred under vacuum to the preparation chamber where the sample was fractured and etched under vacuum. Samples were then sputter coated with gold for imaging. All images were processed and analyzed in ImageJ.

### Fluorescence Molecular Tomography

ELP(V₄A₁) was fluorescently labeled with Alexa Fluor 647 NHS ester with a reaction efficiency of ˜50%. Excess dye was removed by ultrafiltration using Amicon Ultra Centrifugation Filters. Labeled ELP was mixed with unlabeled ELP to obtain a final fluorophore concentration of 1 μM. Prior to injection, POP(V)−25% and ELP(V₄A₁) were endotoxin purified to <1 EU/ml and mixed to a final POP concentration of 250 μM and ELP concentrations of 10 μM, 100 μM, or 250 μM, CS7BL/6J mice at 8 weeks old were shaved below the midline and injected subcutaneously on the right hind flank with 200 μL of the POP-ELP mixture corresponding to the appropriate group, At 0 (immediately after injection), 4, 12, 24, 48, 72, 108, 144, 192, and 240 h post-injection, mice were anesthetized with 2.5% isoflurane and imaged with a Fluorescence Molecular Tomography 4000 In Vivo Imaging System. Quantification of fluorescence in the region of interest was performed using TruQuant software.

### Atomic Force Microscopy

A commercial Asylum MFP-3D system was used for all experiments. All experiments were conducted in PBS at room temperature. Drift was minimized by equilibrating the system at least 15 to 30 min in solution prior to any measurement. 5 μm borosilicate beads were attached to AFM cantilevers with a spring constant 0.58 N/m. An optical microscope and a micromanipulator were used to apply UV cure epoxy and borosilicate beads to tipless cantilevers. After the beads were adhered to the cantilever, they were cured by UV irradiation at 366 nm for 90 min. Excess epoxy was removed by reactive ion etching and serial rinsing in a 1% (v/v) sodium dodecyl sulfate solution in deionized water solution, deionized water, and ethanol. Only attached beads with defect free surfaces, as confirmed by scanning electron microscopy, were used for subsequent AFM measurements. Cantilevers modified with attached beads were functionalized. with a 2 nm Cr layer and a 10 nm Au overlayer by E-Beam evaporation. Coated probes were then incubated overnight with triethylene glycol to form a uniform nonfouling monolayer. The deflection sensitivity was calibrated by engaging the cantilever on a silicon surface in deionized water. The spring constant, k_{c}, of the cantilever was determined from the power spectral density of the thermal noise fluctuations in air by fitting the first free resonant peak to known equations for a simple harmonic oscillator. All data processing and calculations were performed in MATLAB. The contact point between the probe and sample in force curves were identified visually and used to offset the force curves to 0 at the contact point. A Hertzian contact mechanics model was fitted to these curves to calculate the Young's modulus (E). For each individual microparticle, an estimate of particle radius was obtained by Imager analysis of images collected from under the AFM video feed. For planar surfaces, a radius of infinity was used in the model. All samples were adhered to glutaraldehyde activated glass coverslips in PBS and checked under the microscope with gentle agitation to ensure rigid coupling to the underlying substrate.

### Statistics

All statistical analysis was carried out using GraphPad Prism 8. When comparing individual groups, two-tailed t-tests were used to determine statistical significance. ANOVA was used to evaluate significance among three or more groups and with appropriate post hoc tests where indicated in the text for comparisons between groups. For bulk network void volume measurements and particle video analysis, an n=5 was chosen. For particle analysis and measurements, the largest feasible group size was chosen with a minimum n of 50 measurements. Specific experimental group sizes are reported in the description of each experiment. All experiments were repeated at least three times with similar results, and all microparticle images are representative of their broader population. Polymers were purified several times from independent stocks to ensure observed behavior was not batch dependent.

### Example 2

### Complex Microarchitectures from Stimuli-Responsive Intrinsically Disordered Proteins

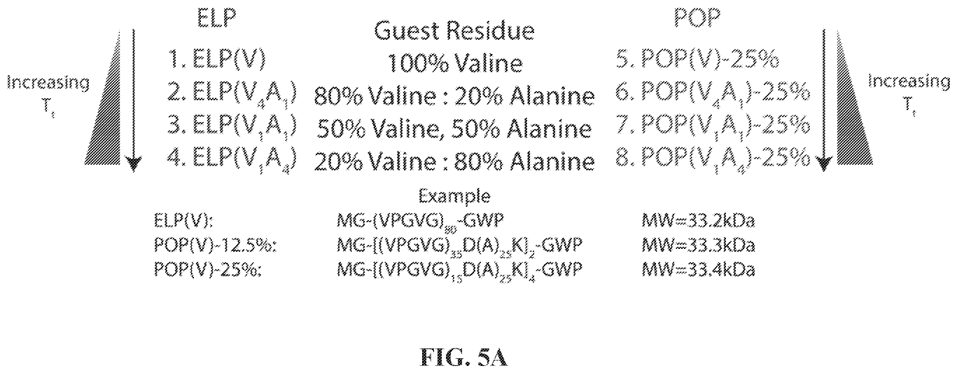

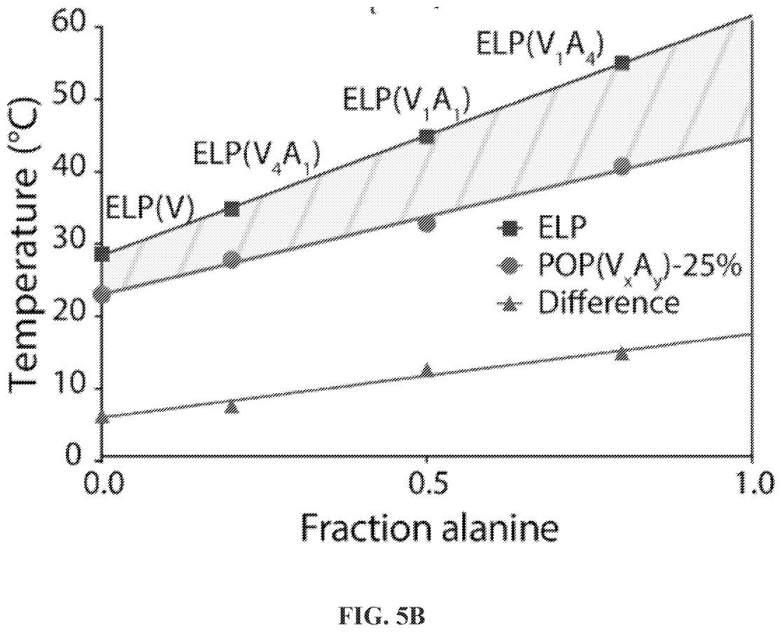

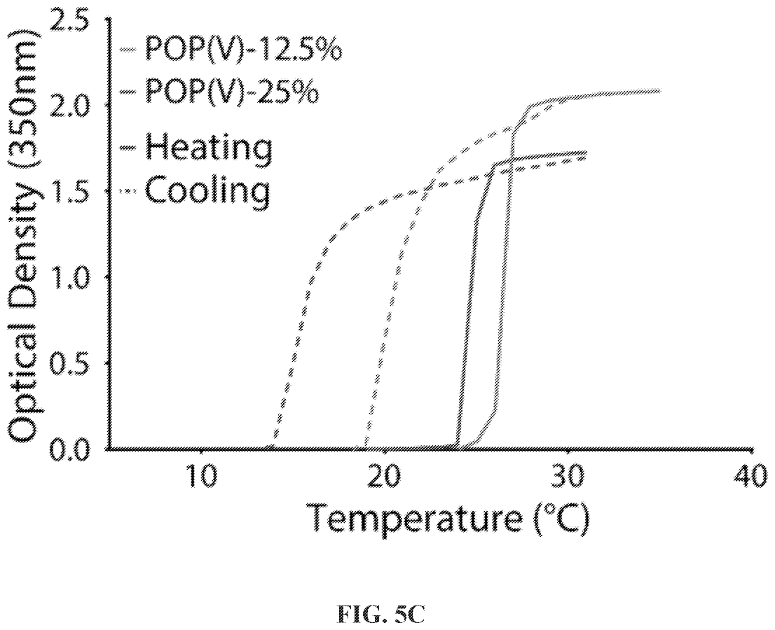

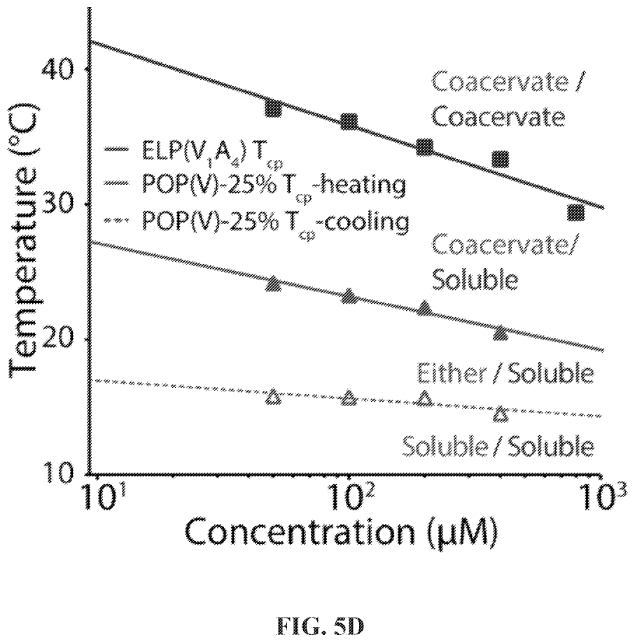

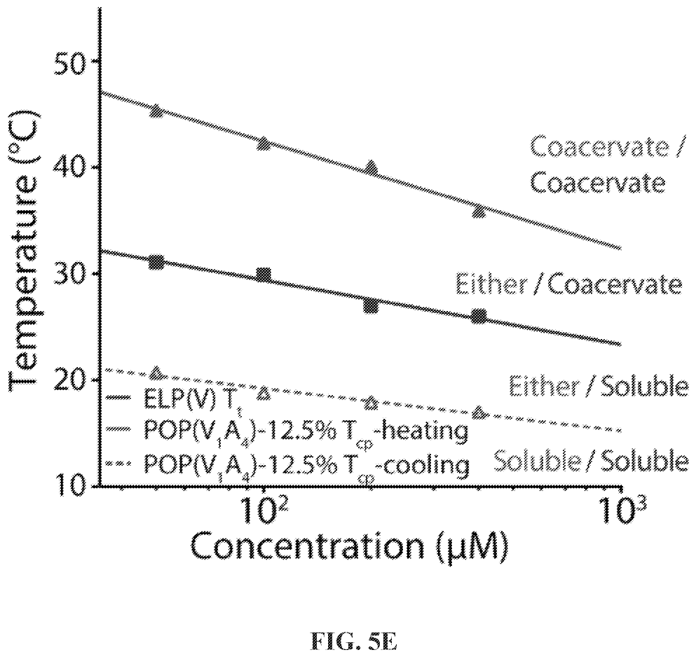

The T_{cp} of POPs and ELPs are tunable by the identity and mole fraction of the guest residue (X) in the VPGXG repeat unit, and the T_{cp}'s of POPs are further tunable by the mole fraction of embedded oligoalanine helices. We therefore used different ratios of alanine (A) and valine (V) in the guest residue position in ELPs and POP and the helical content of POPs to span a range of T_{cp}'s of ELPs and POPs from 20° C. to 50° C. (FIG. 5). This range of T_{cp}'s allows us to use two distinct types of POP-ELP mixtures: (1) one in which the POP is designed to transition at a lower temperature than the ELP upon heating the mixture, and (2) another in which the POP is designed to transition at a higher temperature than the ELP. Within POP-ELP mixtures, the two IDP populations are not fully miscible, with distinct aggregation events observed for both populations.

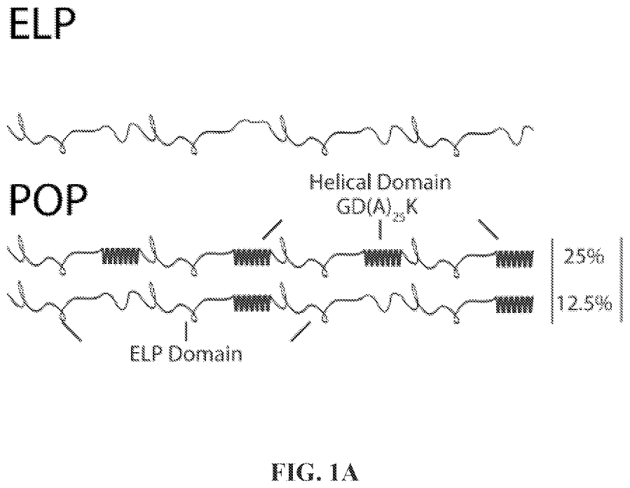

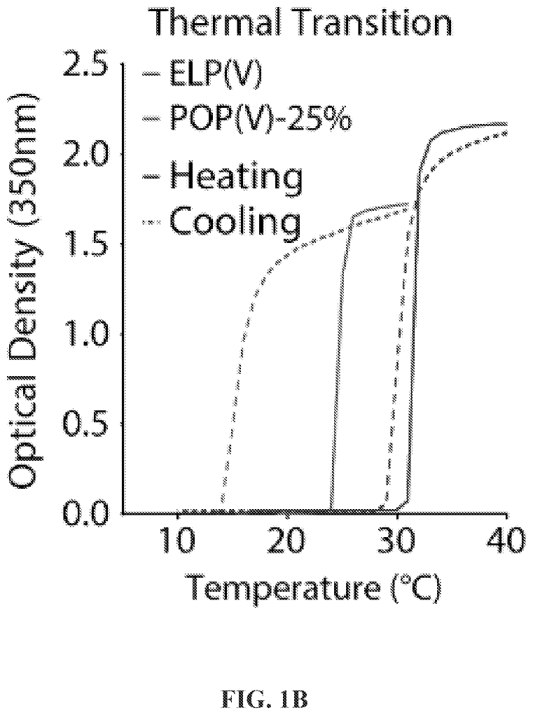

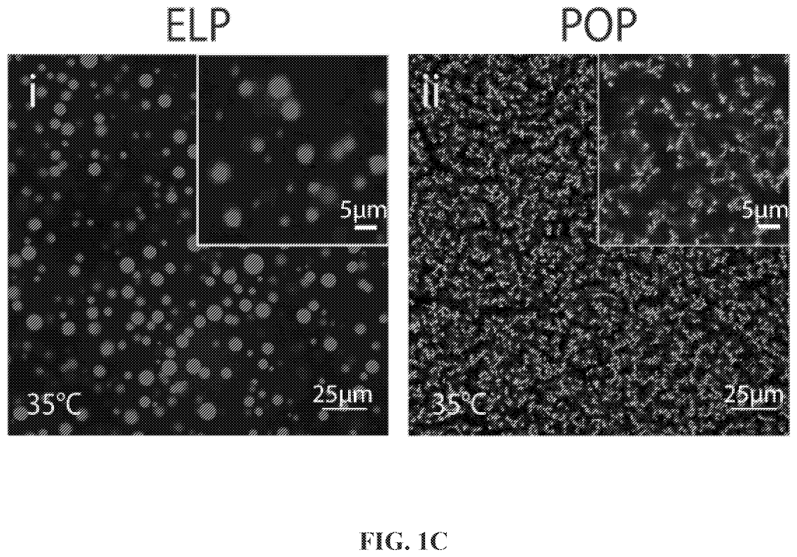

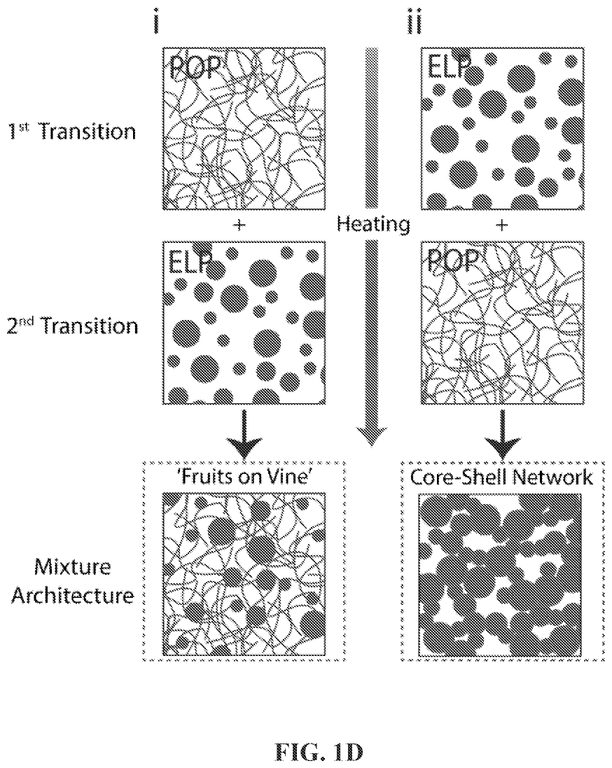

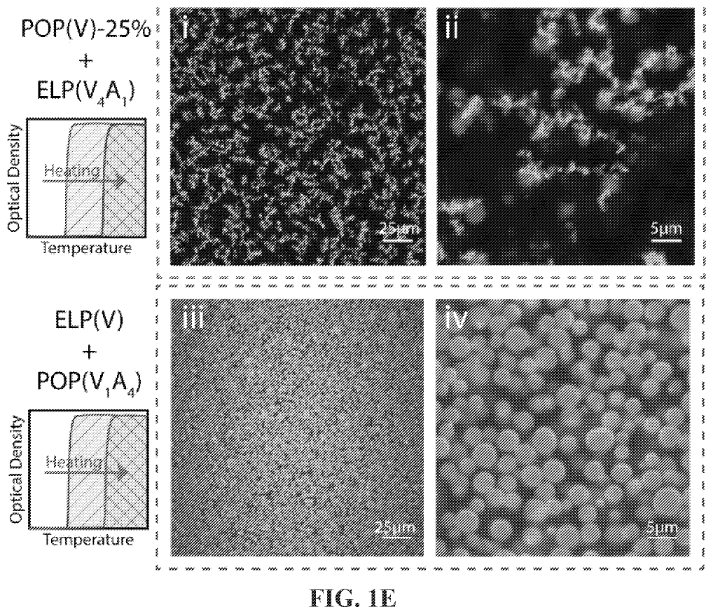

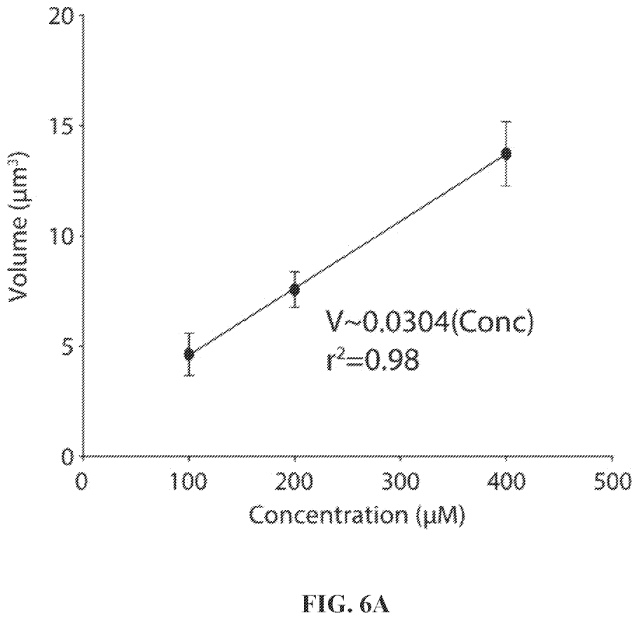

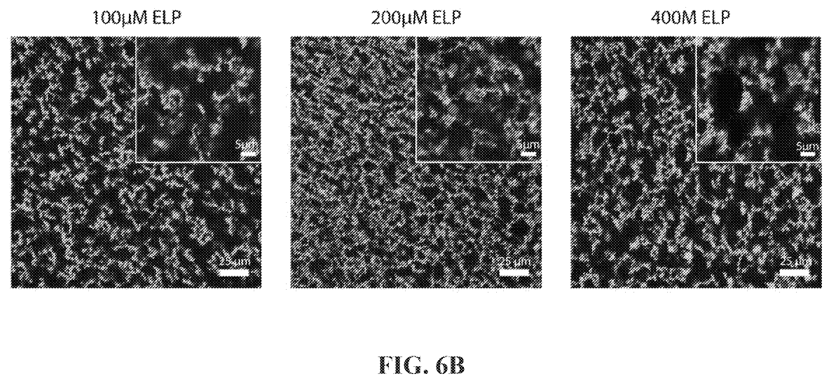

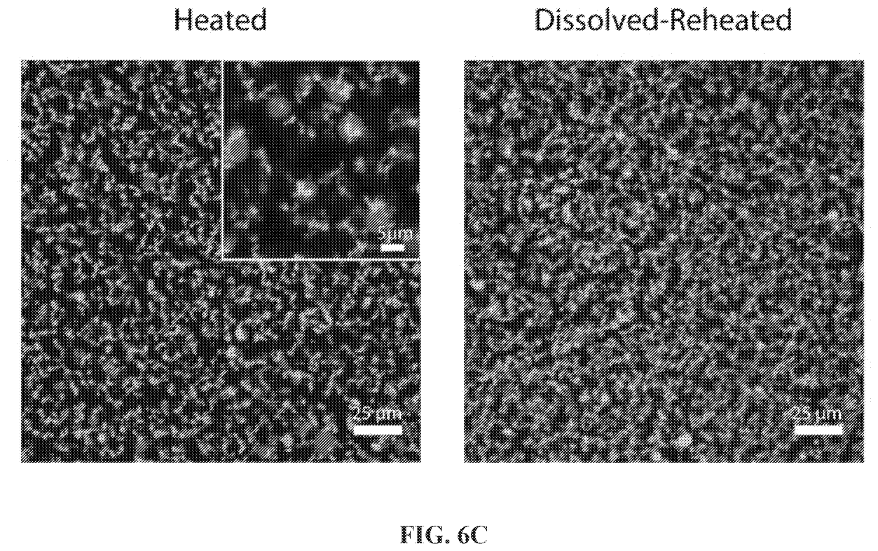

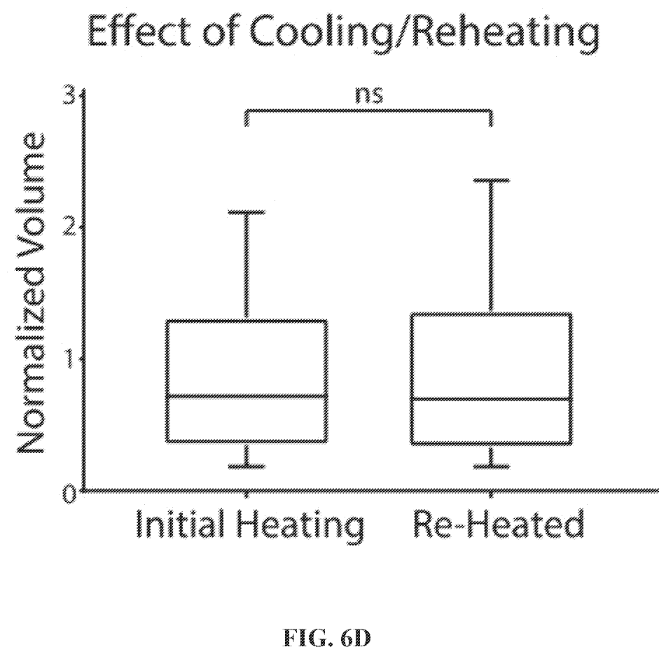

To demonstrate the structural consequence of phase separation in system 1, we used a mixture of POP(V)−25% (where ‘V’ designates the guest residue amino acid and 25% designates the fraction of oligoalanine) with ELP(V₄A₁). Above the T_{cp}-heating of POP(V)−25%, the IDP forms a stable, porous network. Continued heating to above the T_{cp} of the ELP causes ELP coacervates to form and grow until they are able to interact with the preformed POP network. Upon contact with the network, they become immobile, forming ‘fruits’ of ELP on a POP network ‘vine’ in solution as illustrated in the schematic (FIG. 1D) and confocal microscopy sections (FIG. 1E). While the size of the ELP globules is somewhat varied, the average volume of the globules correlates with the concentration of ELP in solution without altering the POP network structure (FIG. 6). The system can also be cooled below the ELP T_{cp}—but above the T_{cp}-cooling of the POP—and reheated with no change in the average globule size (FIG. 6).

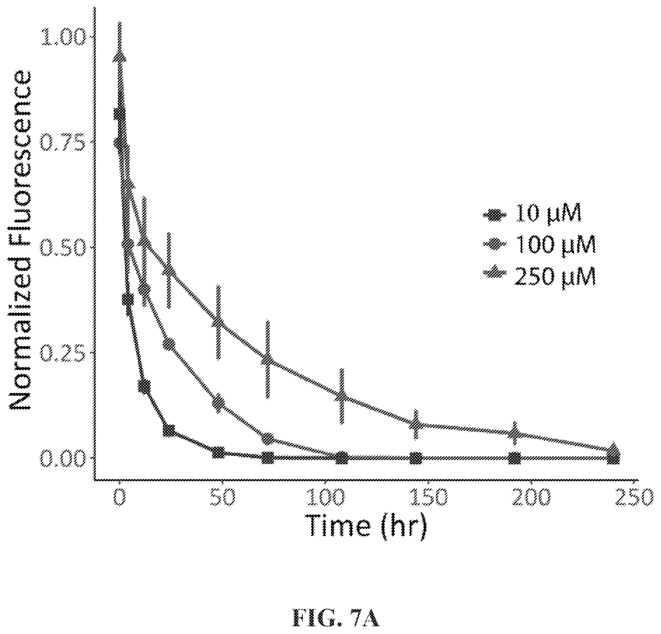

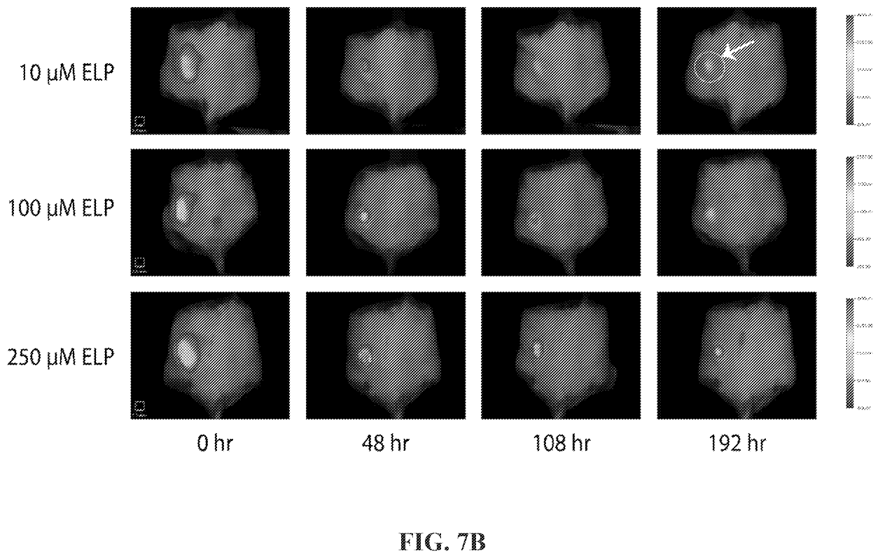

System 2, with ELP(V) designed to coacervate before POP(V₁A₄)−25%, forms a different type of structure. Heating above the T_{cp}, of the ELP forms polymer coacervate droplets. Upon raising the temperature above the T_{cp}-heating of the POPs, however, the POPs do not form a macroporous network, but instead wet with the outer edges of ELP coacervate droplets and form a physically crosslinked interconnected porous shell, such that the structure includes spherical ELP coacervate droplets encased in a lattice-like shell of the POP coacervate as illustrated (FIG. 1D) and shown in confocal microscopy sections (FIG. 1E). Due to the hysteretic nature of POPs and their significantly lower T_{cp}-cooling than T_{cp}-heating, subsequent cooling of the system to T<T_{cp} of the ELP but T>T_{cp}-cooling of the POP dissolves the ELP cores into the aqueous phase through the pores of the POP shell, and creates an interconnected network of hollow protein shells. These types of two-protein systems are a simple way to create a drug eluting scaffold using a single injectable system. To demonstrate proof-of-concept of this approach, we used fluorescence molecular tomography (FMT) to monitor the release of ELP(V₄A₁) co-injected with POP(V)−25% in the subcutaneous flank of mice (FIG. 7). The ELP “fruits” that hang from the POP “vine” slowly dissolve and are secreted out of the POP scaffold over approximately 10 days without affecting the size of the POP scaffold. The release kinetics are further tunable with ELP concentration without altering the concentration of the co-injected POP.

### Example 3

### Microdroplet Architecture

Given the limited available architectures for biomaterial microparticles and the ease of formation for these atypical POP-ELP architectures in bulk, we next sought to translate these structures to microscale droplets. To do so, we used a microfluidic emulsion droplet generator in a T-junction design (FIG. 2A) capable of producing highly monodisperse water-in-oil emulsion droplets (FIG. 2B).

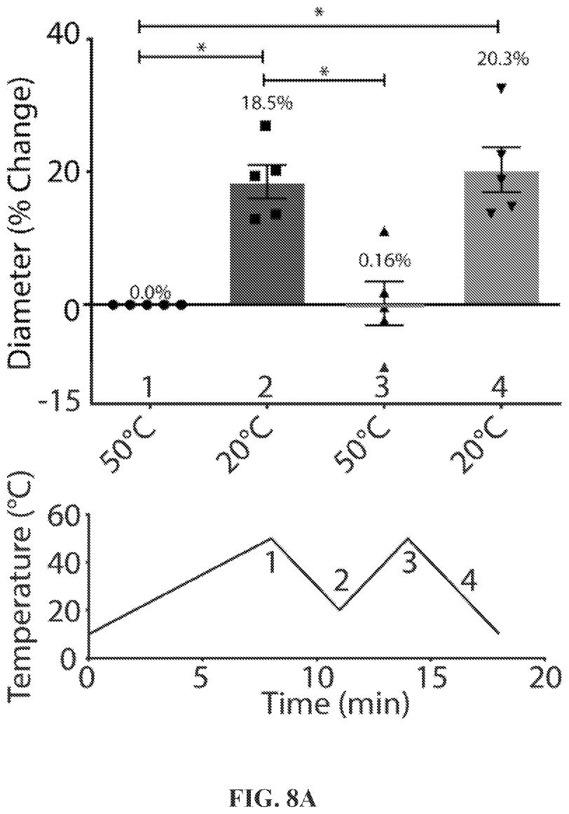

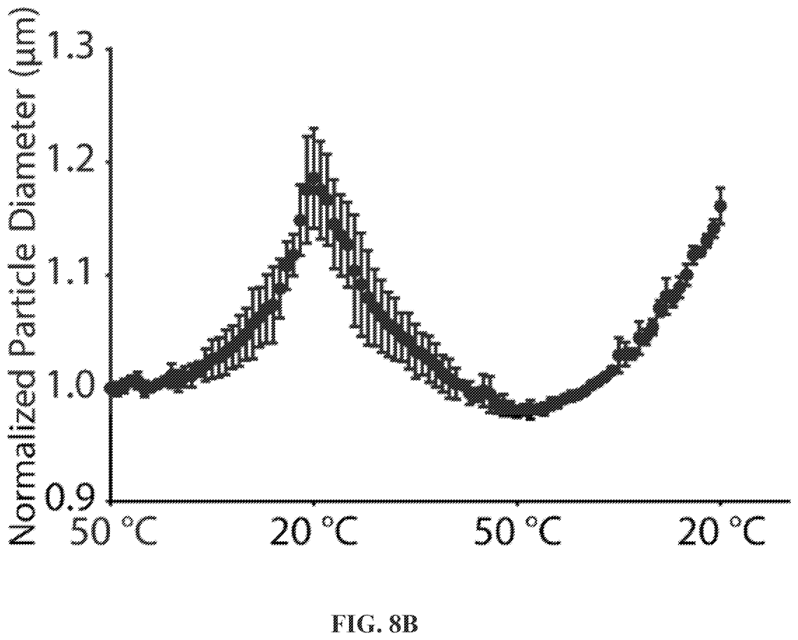

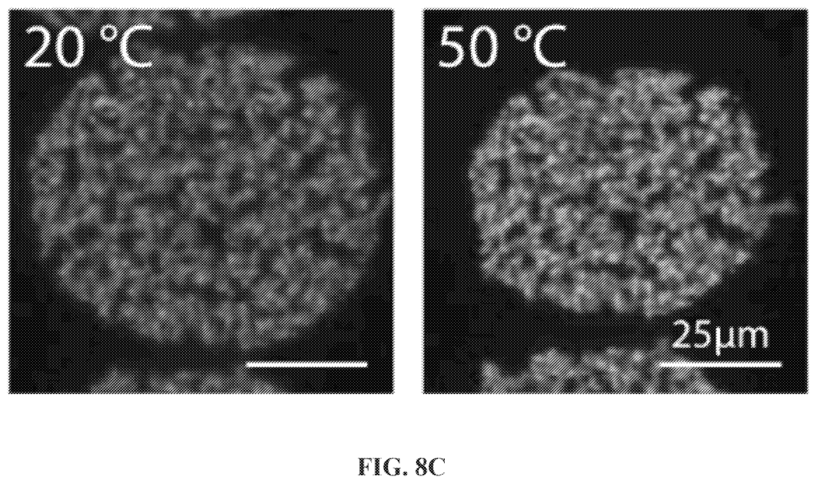

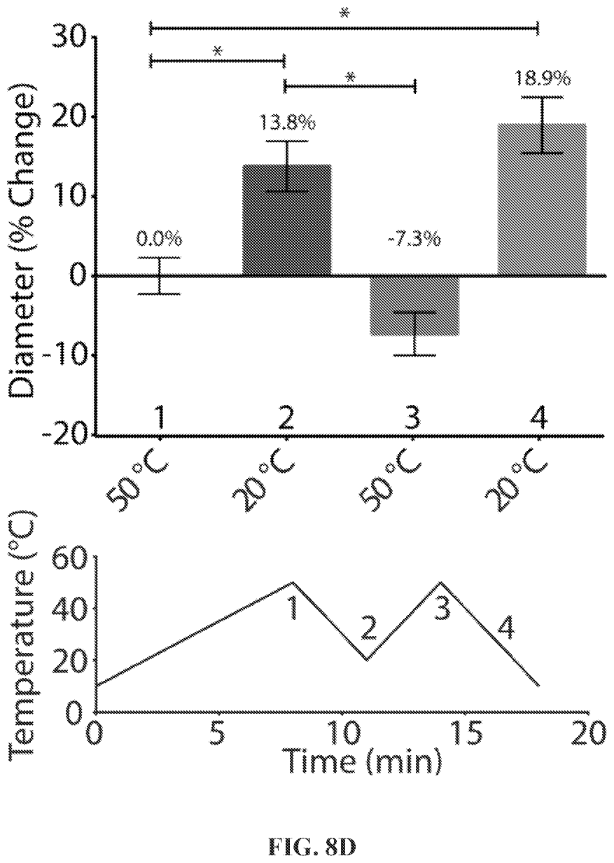

The ELP and POP components in PBS were premixed and the entire device was kept at 4° C. during droplet generation to ensure uniform distribution of the soluble IDPs within each droplet. We controllably triggered subsequent ELP and POP phase transitions within the microdroplets by heating and cooling to generate a range of coacervate microstructures. We first examined the structures formed solely by POP(V)−25% in microdroplets, and found that the POP produces stable structures with microarchitectures similar to those observed in bulk (FIG. 2C, FIG. 2D, and FIG. 2E), forming fractal-like porous microparticles with high void volume above the T_{cp}-heating. Continued heating and cooling above the aggregation temperature leads to nonlinear shrinking and swelling of the microparticles (FIG. 8). Heated particles shrink/swell by as much as 20% between 20° C.-50° C., and the process is fully reversible. POP(V)−12.5% can also be used, forming similar microparticles with only a slightly higher T_{cp}-heating than the 25% POP. Particles remain stable when cooled into the meta-stable hysteretic temperature range, and subsequent cooling below the T_{cp}-cooling of the POP results in dissolution of the microstructure.

We next included ELP as a second polymer component within the aqueous phase of the microfluidic set-up to recapitulate the unique architectures seen in bulk within a confined microenvironment with the end goal of creating microparticles with unique morphologies and internal microstructures. Mixtures of POP(V)−25% and ELP(V₄A₁), in which the POP phase separates at a lower temperature than the ELP, were thermally programmed into the five distinct states seen in the overlaid ELP and POP phase diagram (FIG. 2F. FIG. 2G, and FIG. 2H): (1) ELP and POP are soluble; (2) upon heating to a temperature>T_{cp}-heating of the POP, the POP phase separates and forms a porous microparticle; (3) upon continued heating to T>T_{cp} of the ELP, the ELP coacervates into immiscible globules that wet the POP network; (4) upon cooling below the T_{cp} of the ELP, the ELP dissolves; and (5) and further cooling to T<T_{cp-cooling} of the POP cooling also re-dissolves the POP.

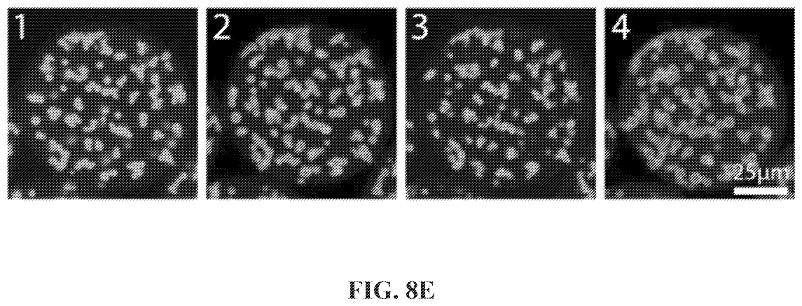

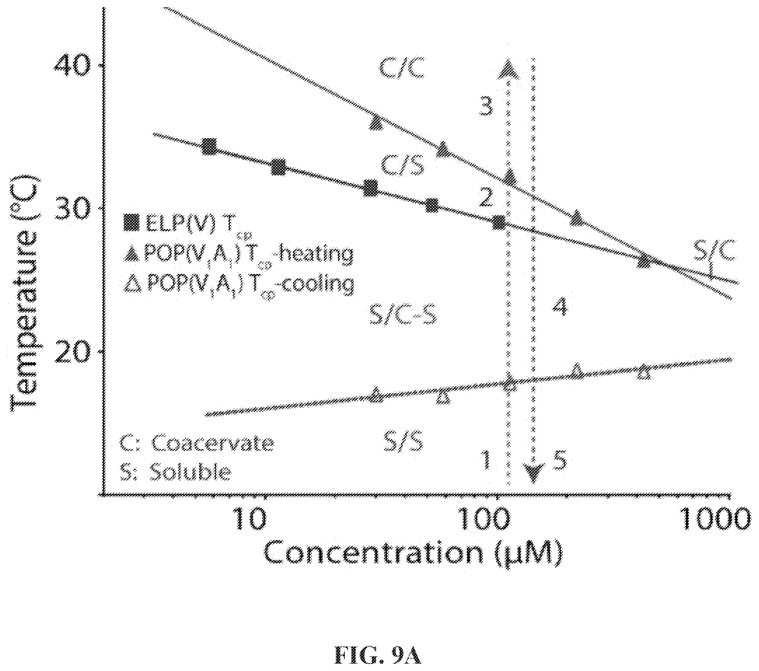

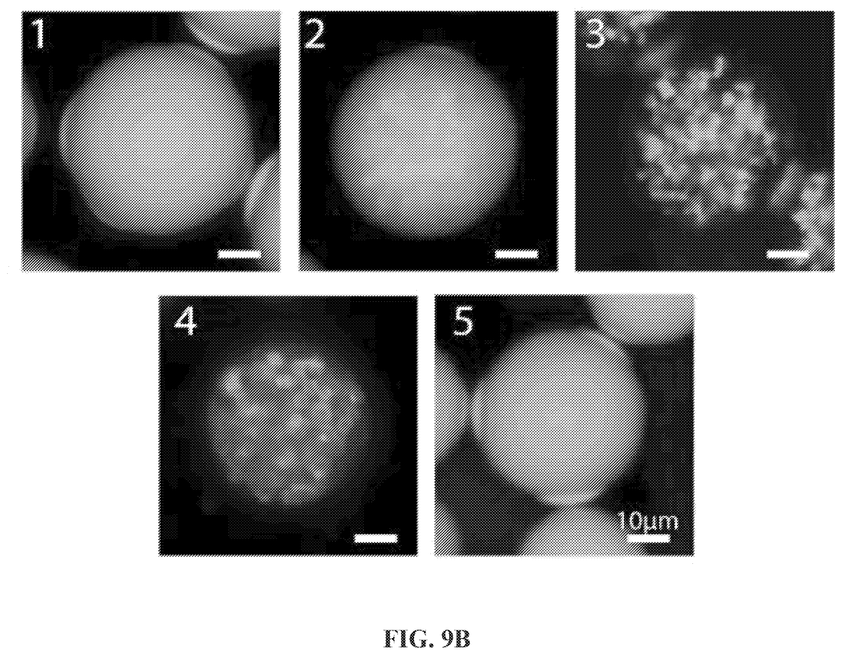

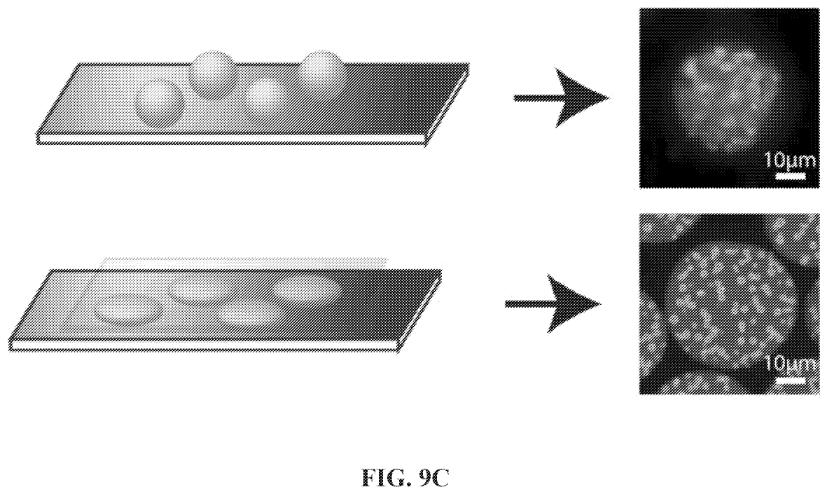

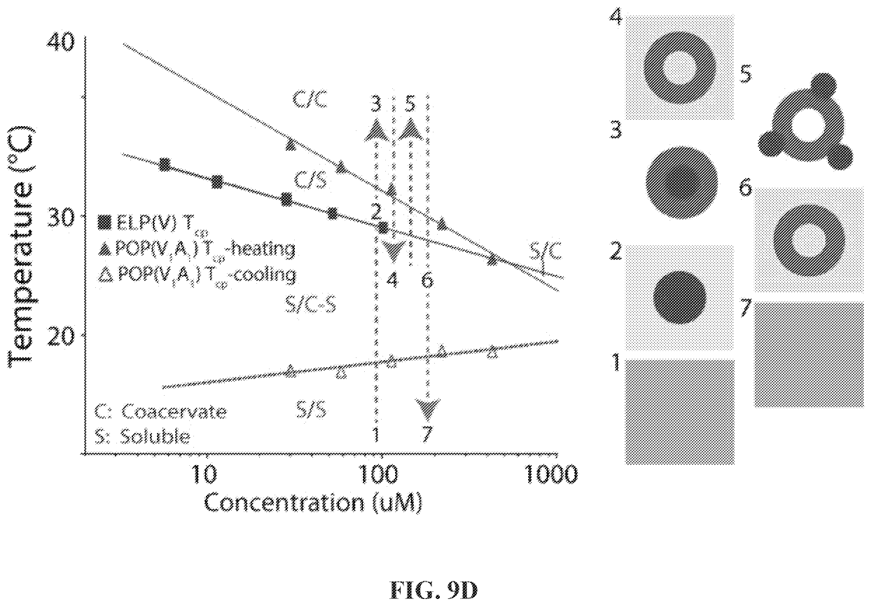

In contrast, mixtures of ELP(V)+POP(V₁A₄)−25% in which the ELP coacervates at a lower temperature than the POP (T_{cp}ELP<T_{cp}-heating POP), can be cycled through core-shell and hollow shell networks (FIG. 2I, FIG. 2J, and FIG. 2K) as follows: (1) both IDPs are dissolved; (2) as the temperature is raised such that T>T_{cp}, of the ELP, the ELP coacervates into aqueous immiscible droplets; (3) raising the temperature to T>T_{cp}-heating of the POP triggers the phase separation of the POP, leading to the formation of a conformal porous POP shell on the ELP core; (4) upon cooling to T<T_{cp} of the ELP but T>T_{cp}-cooling of the POP, the ELP dissolves resulting in a network of hollow POP shells; and (5) finally, as the temperature is lowered below the T_{cp}-cooling of the POP, the POP re-dissolves, fully restoring the system to its original state of a mixture of soluble ELP and POP. Like the porous POP microparticle networks, the hollow POP shells also swell and shrink ˜20% in size when heated and cooled after formation (FIG. 8). After reaching state 4, where the ELP has dissolved out from within the POP shells into the aqueous phase of the droplets, leaving behind intact porous POP shells, if the temperature is then raised above the T_{cp} of the ELP, the ELPs will re-coacervate, forming aqueous-immiscible ELP globules that wet the outside of the hollow POP shells (FIG. 9).

### Example 4

### Crosslinked Structures

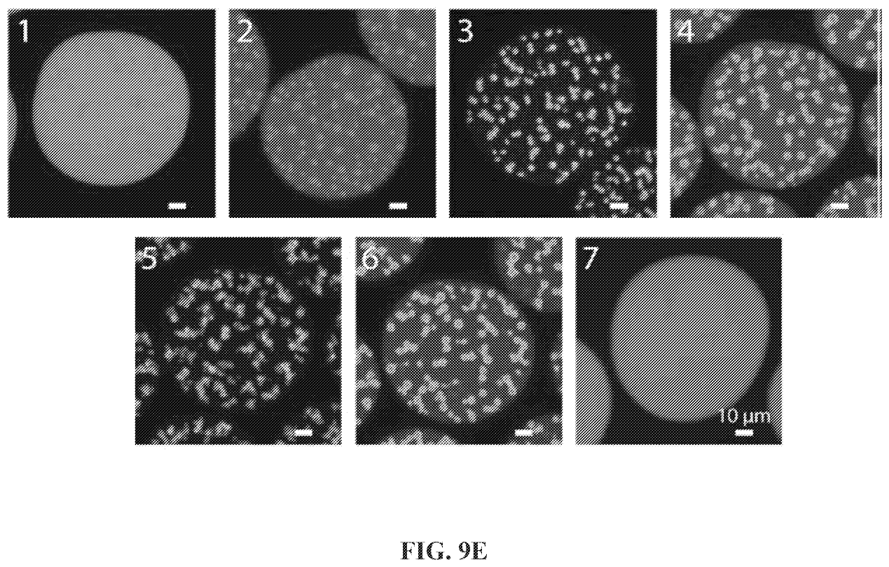

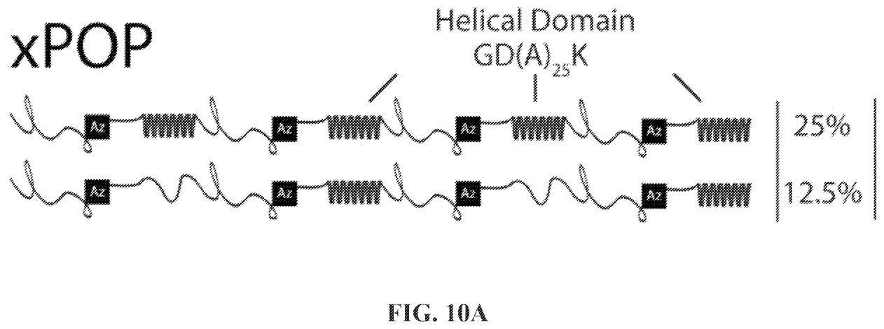

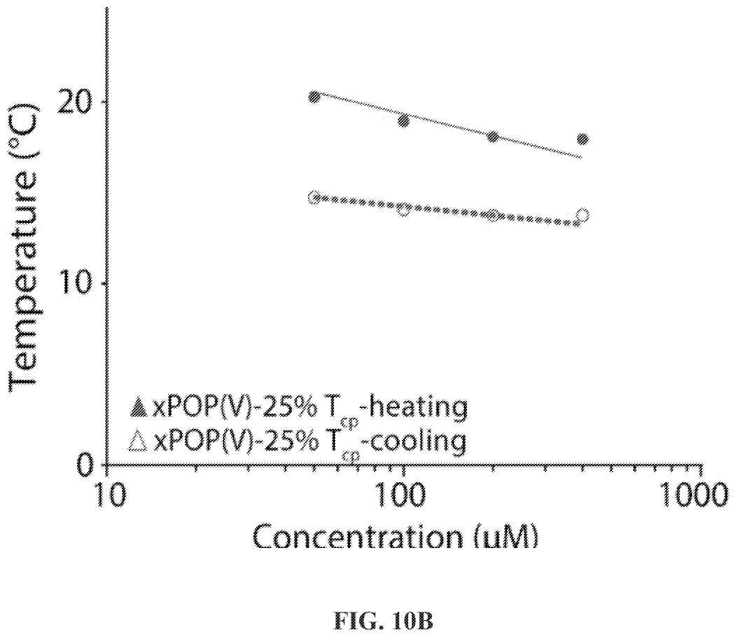

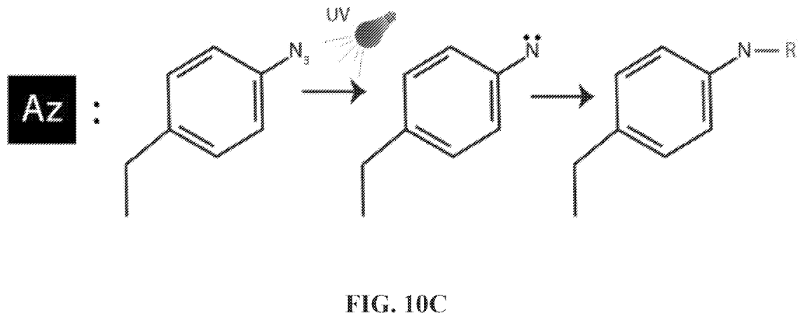

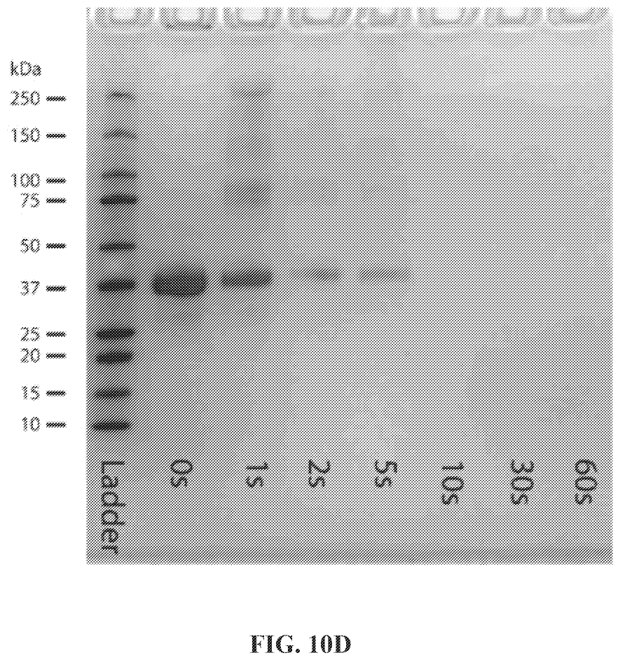

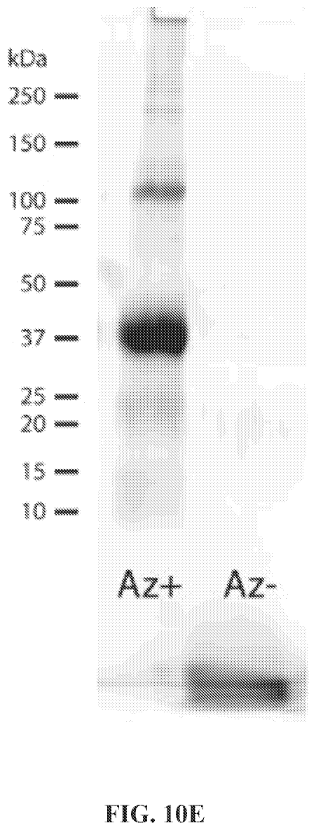

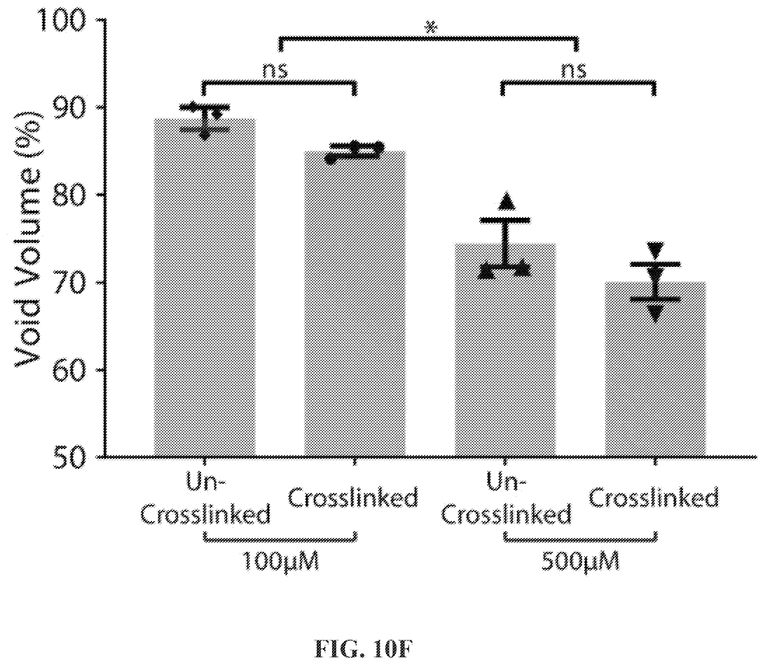

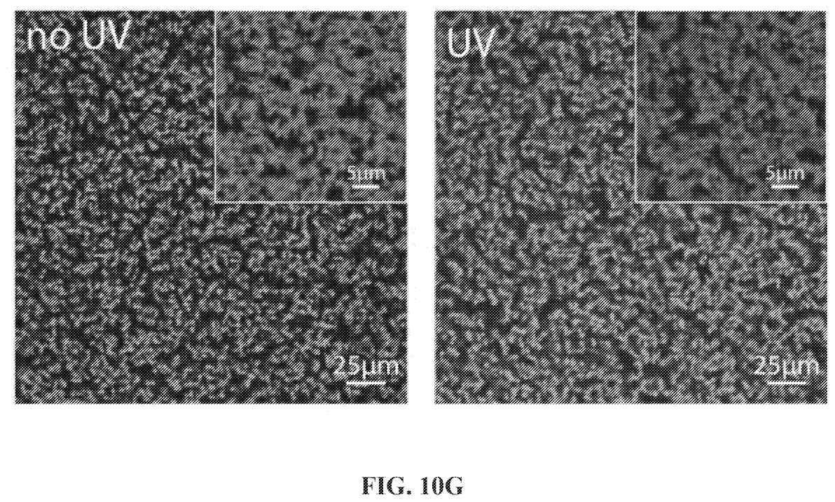

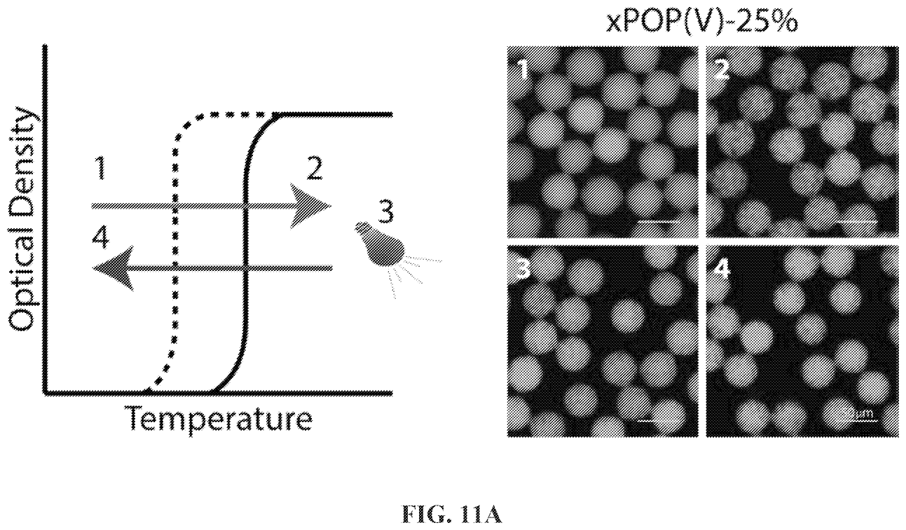

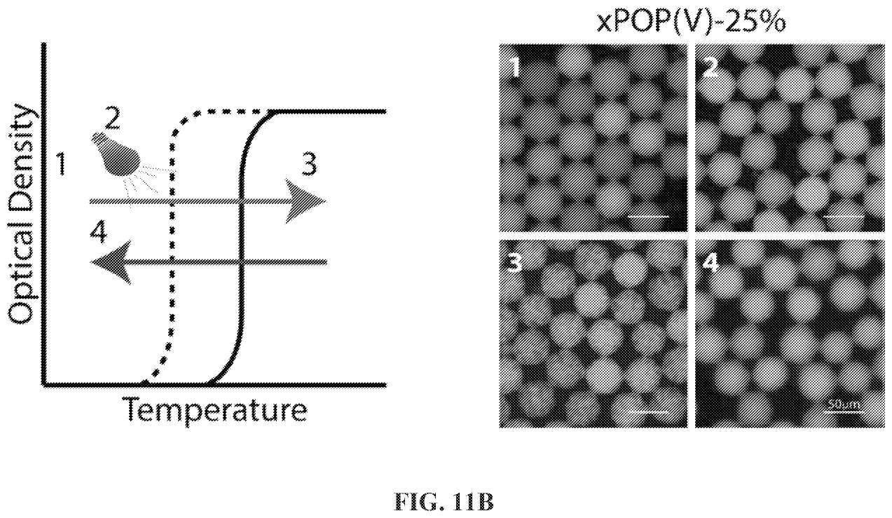

To augment their stability, we next devised a method to crosslink the microstructures without the need of extrinsic crosslinking agents, and without the formation of potentially toxic byproducts—even though such crosslinking methods could be used for these structures and polypeptides. To do so, we pursued multi-site unnatural amino acid (UAA) incorporation of para-azidophenylalanine (pAzF), which participates in a host of crosslinking reactions following exposure to ultraviolet (UV) light (see Chin, J. W. et al. Addition of p-azido-1-phenylalanine to the genetic code of Escherichia coli. Journal of the American Chemical Society 124, 9026-9027, (2002), which is incorporated by reference herein in its entirety). A small library of crosslinkable xPOPs was created and their thermal and microarchitecture properties were characterized (FIG. 10). xPOPs are similar to POPs in their sequence, with the exception that pAzF residues are equally spaced throughout the polymer at 1 pAzF per 100 residues. While the T_{cp}-heating and T_{cp}-cooling of the xPOPs are slightly depressed relative to the parent POP due to the hydrophobicity of pAzF, the xPOP microemulsions undergo the same coacervation process, forming porous microparticles. The xPOPs photochemically react after only short UV exposure, requiring <10 s of exposure time to fully crosslink. Network architecture and void volume in bulk are unaffected by crosslinking (FIG. 10), When crosslinked above their T_{cp}-heating, subsequent cooling to below T_{cp}-cooling does not resolubilize the microparticles, unlike their non-crosslinked counterparts (FIG. 11).

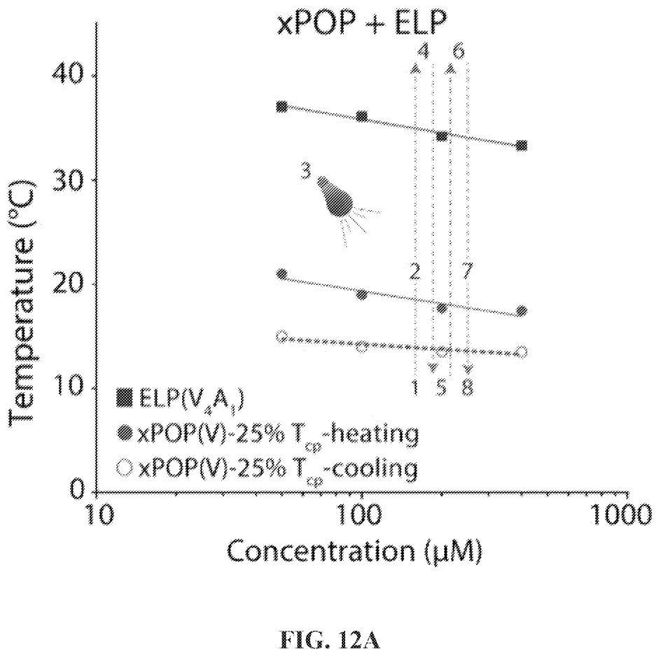

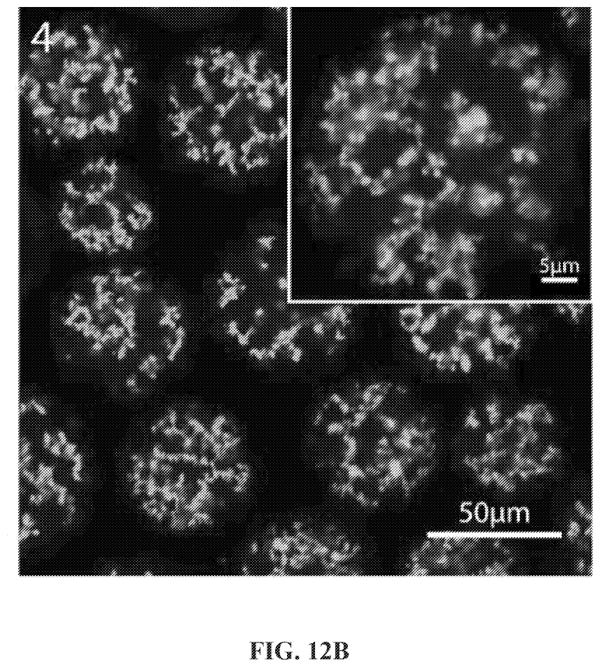

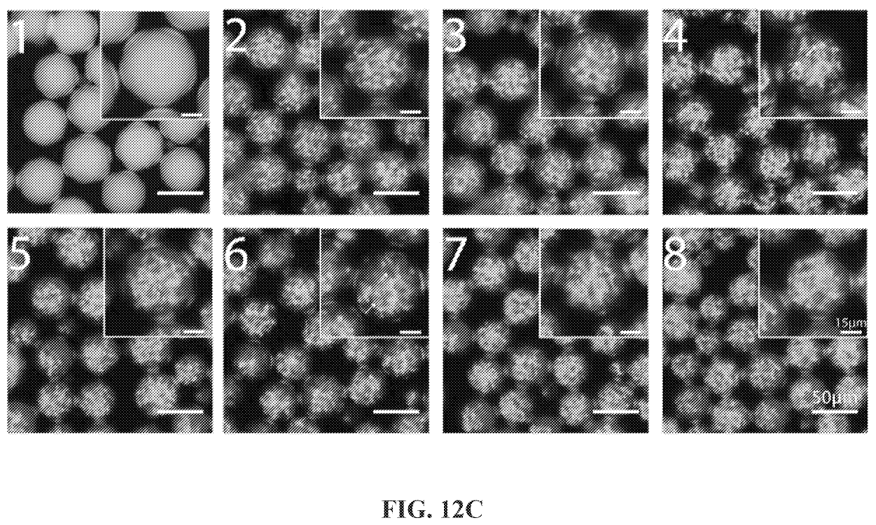

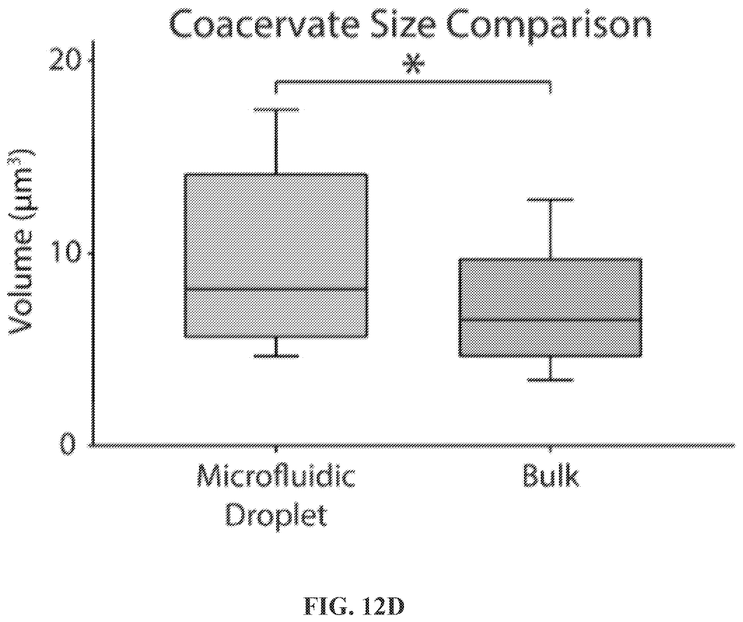

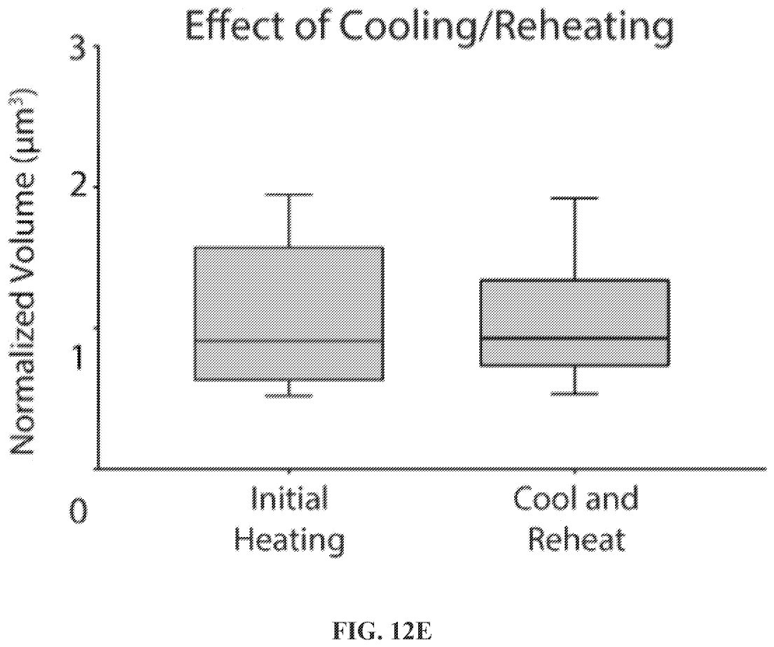

xPOPs can also be readily mixed with ELPs to stabilize microparticle architectures. Mixtures of xPOP(V)−25% and ELP(V₄A₁) form a fruits-on-a-vine architecture when heated above the IDPs' aggregation temperatures similar to that previously observed for a POP and ELP mixture (FIG. 12). Subsequent exposure to UV light crosslinks the POP, allowing the ELP to solubilize, but preventing the POP from solubilizing even when cooled well below its T_{cp}-cooling. If the system is re-heated, ELP globules reform, and this process can be repeatedly cycled without altering the POP microparticle architecture. Cooling and re-heating does not alter ELP globule properties, and the average “fruit” size remains unchanged. Compared to bulk mixtures, the ELP “fruits” formed in microparticles are similar in shape but slightly larger in size (p<0.05, Student's t-test, n=50).

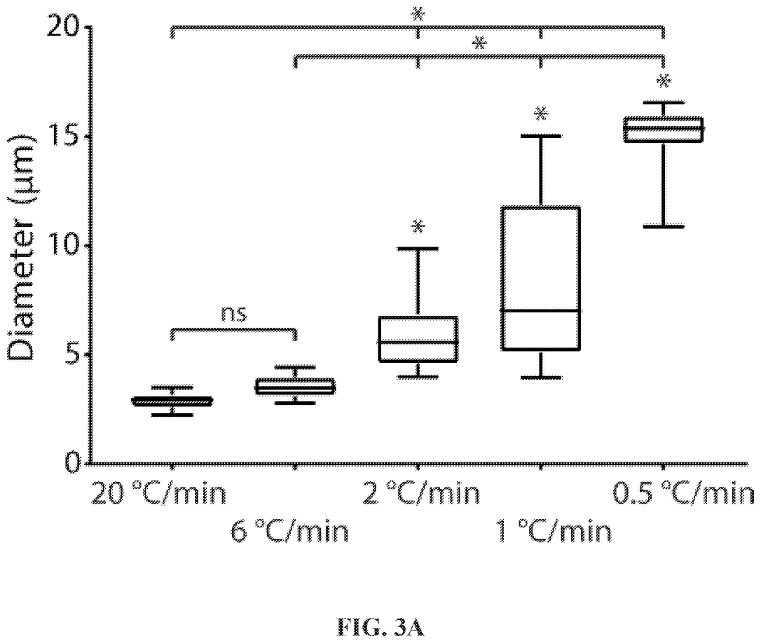

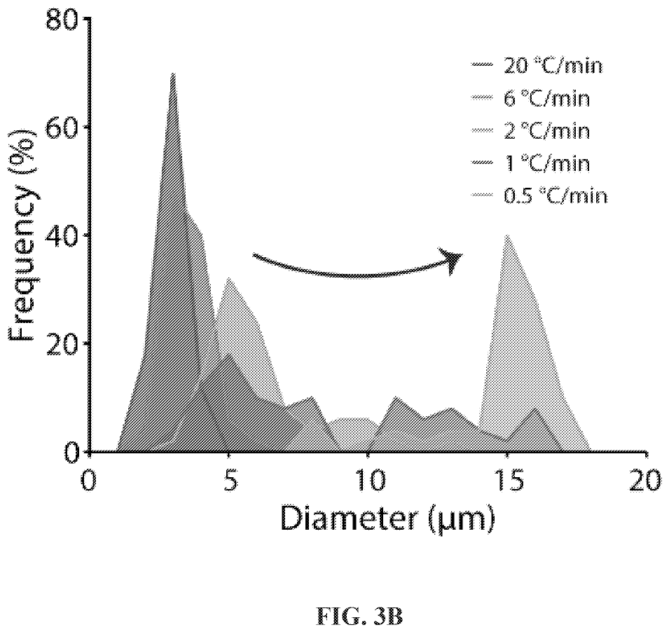

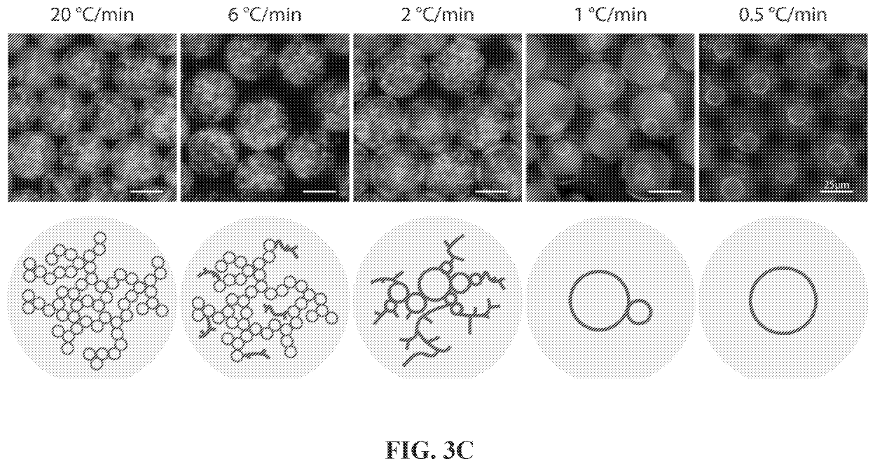

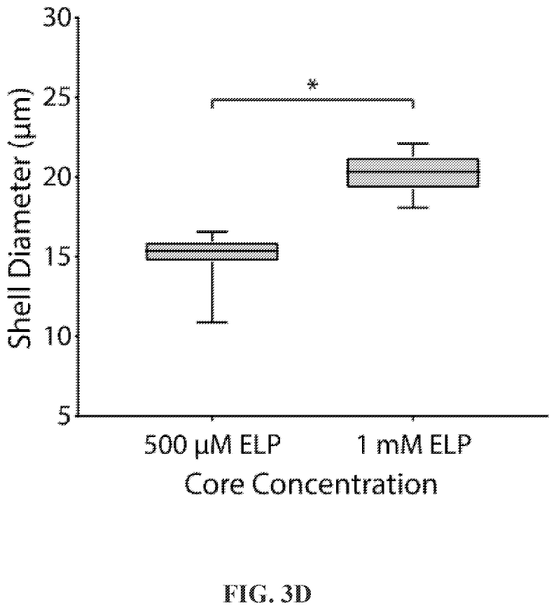

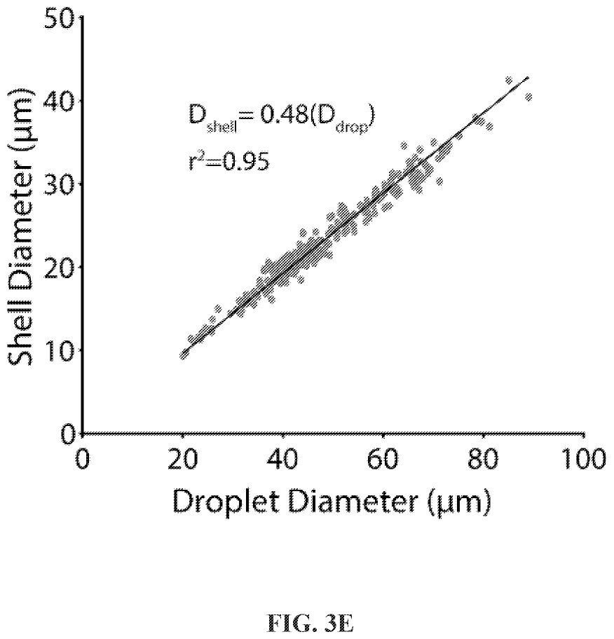

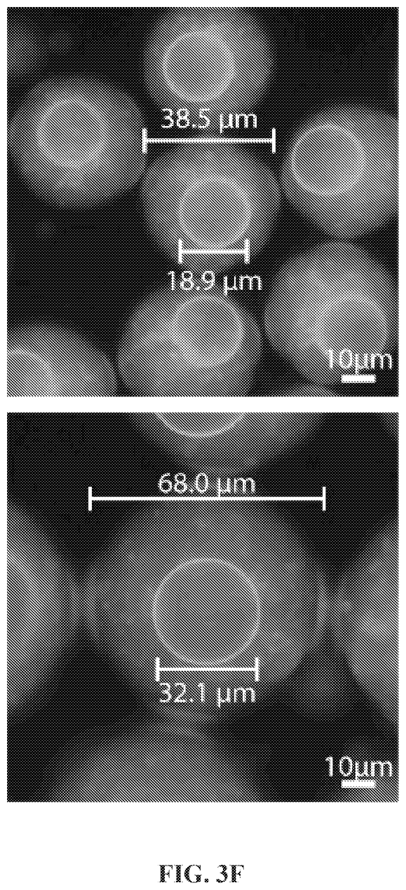

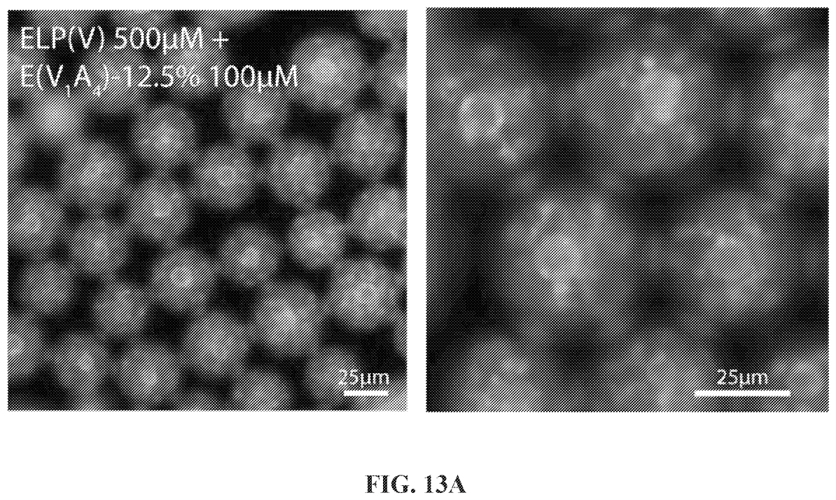

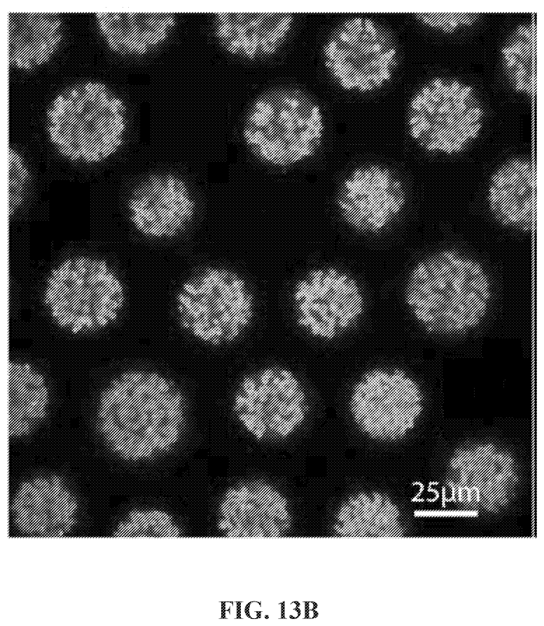

Within the polymer sequence framework, the order in which the two components—ELP and POP—phase separate controls the type of architecture that is formed, rather than the specific sequences of the chosen POP and ELP. For example, mixtures of ELP(V)+POP(V₁A₄)−12.5% (FIG. 13) and ELP(V)+POP(V₁A₁)−25% (FIG. 9) both form core-shell structures similar to ELP(V)+POP(V₁A₄)−25% (FIG. 3) despite the differences in all three POP sequences. However, these structures are not wholly identical.

We determined that the smaller the gap in transition temperatures between the ELP and POP, the smaller the resulting individual core-shell structures that make up the network. This observation highlights a useful difference between the combination of non-hysteretic ELP and hysteretic POP and dual emulsion ELPs. Given sufficient time, two immiscible ELPs with different transition temperatures will phase separate from one another into identical structures regardless of when the second ELP transition is triggered. In the ELP-POP system, increasing the temperature range of thermal hysteresis—the T_{cp} gap—also increases the amount of time that ELP is given to coalesce at a constant thermal ramp rate, prior to entrapment by POPs. Given the sequence homology between the disordered components of POPs and ELPs, POPs prefer to aggregate around the ELPs, and once even a very small layer of POP has formed around the ELP, the ELP coacervate droplets can no longer continue to coalesce.

### Example 5

### Controlling Structure Architecture

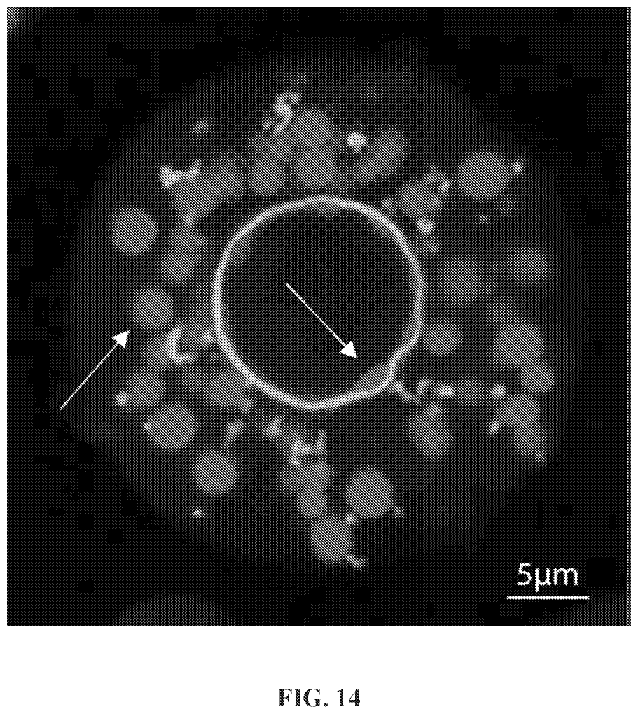

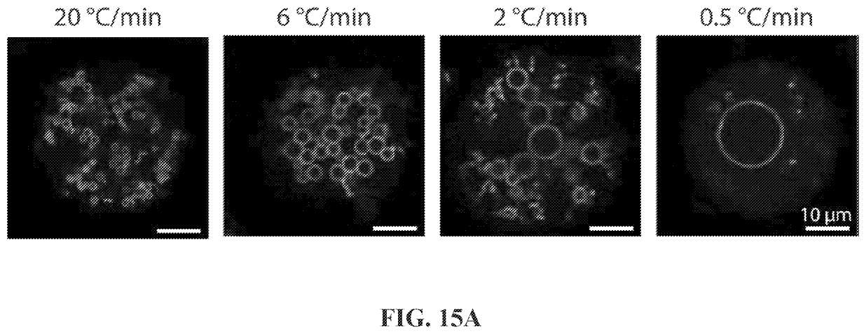

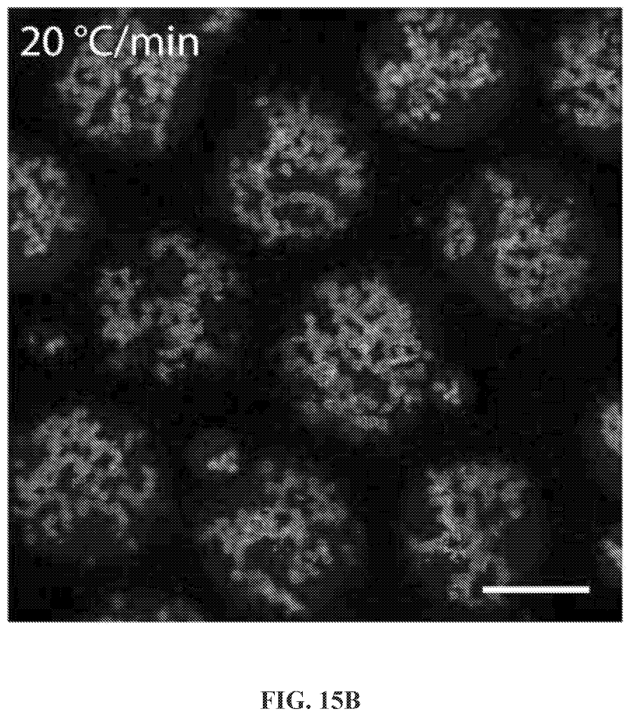

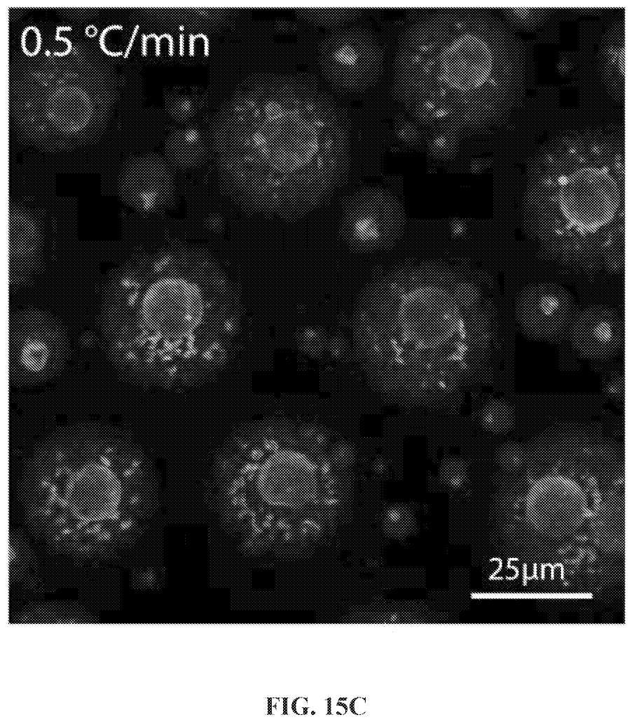

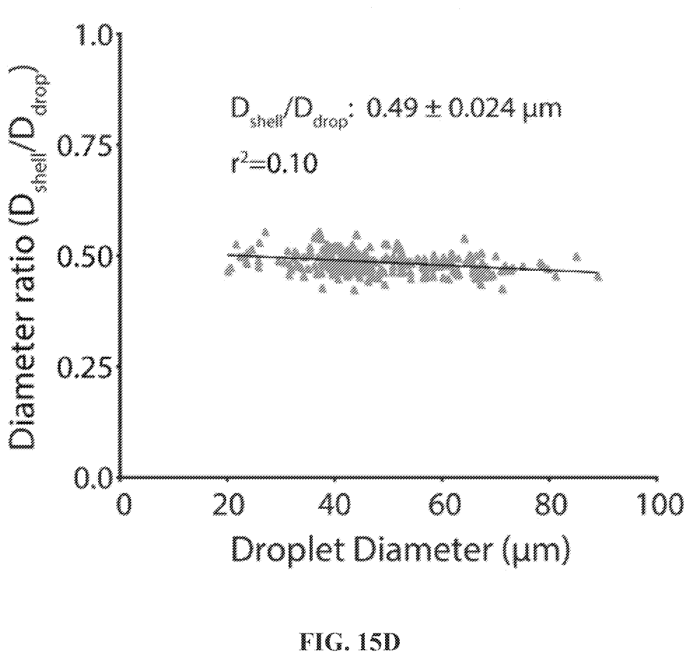

These results suggested that the core-shell architecture can be controlled by heating ELP and POP mixtures at different rates. To investigate this, we used mixtures of ELP(V) and xPOP(V₁A₄)−12.5% to illustrate the spectrum of core-shell structures achievable (FIG. 3A, FIG. 3B, FIG. 3C, and FIG. 15). ELP+xPOP mixtures were heated at ramp rates of 0.5-20° C./min in a thermocycler from 4 to 50° C. and were then UV-crosslinked at the final temperature. Samples were then cooled and transferred to a fluorescent microscope for imaging. At high ramp rates, the ELP is given limited time to coalesce, resulting in disperse ELP globules that become encapsulated by conformal porous shells of the POP, producing a large network of small crosslinked shells. Slightly faster ramp rates produce similar networks, with slightly larger POP shells and some “network-like” arms likely due to (a) the absence of ELP and (b) insufficient time to interact with already aggregated POP. At 1° C./min, a bimodal distribution emerges with an average of one large and one small shell per aqueous droplet. Notably, when the heating rate is slowed to 0.5° C./min, a single hollow-spherical POP shell per droplet is formed. Reheating does not re-fill the shells (FIG. 14), but it does cause aggregation of ELP within and outside of the hollow spheres.

Not only can the architecture of these POP shells be controlled, from a network or hollow protein shells to a single hollow protein shell, we can also control their size (FIG. 3D, FIG. 3E, FIG. 3F, and FIG. 15). If the diameter of the aqueous droplet is kept constant, doubling the volume of ELP within each aqueous droplet, for example, also doubles the volume within a POP shell. The diameter of the droplet provides a more convenient way of controlling size. By varying the speed of the aqueous phase during droplet generation, and therefore creating a polydisperse mixture of droplet sizes, we were able to linearly correlate the diameter of the resultant POP shell to the droplet diameter. With 1 mM ELP(V) as the core-forming component, the diameters of the POP shells were consistently half that of the particle diameter.

### Example 6

### Aqueous Extraction of Structures

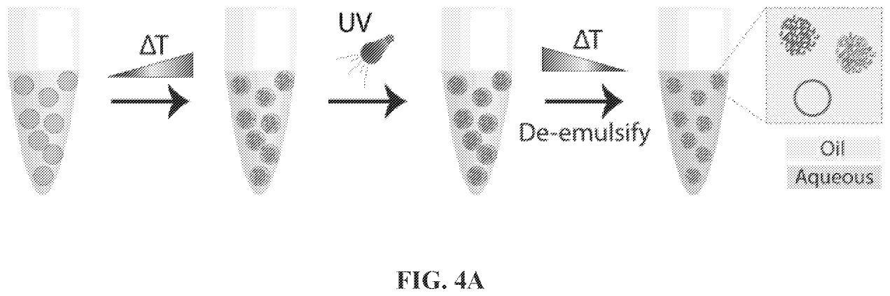

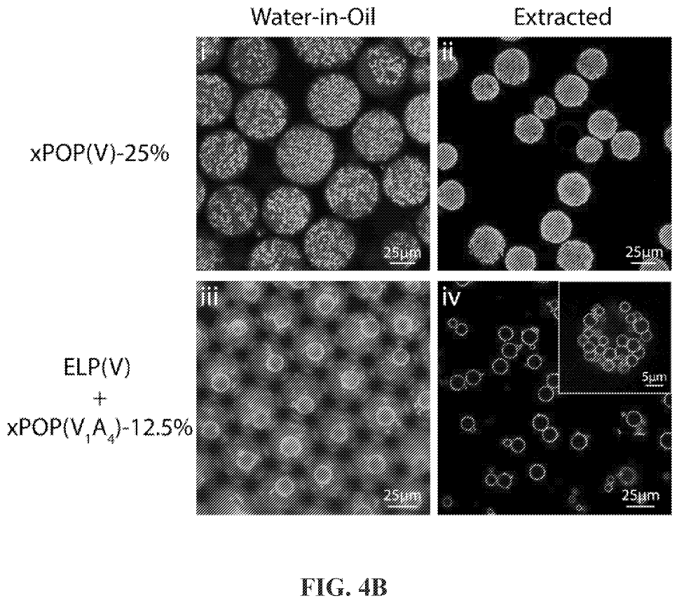

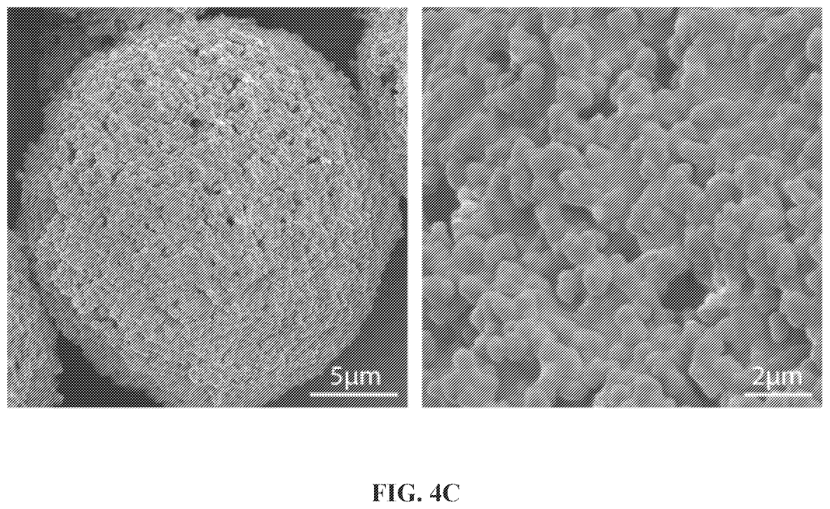

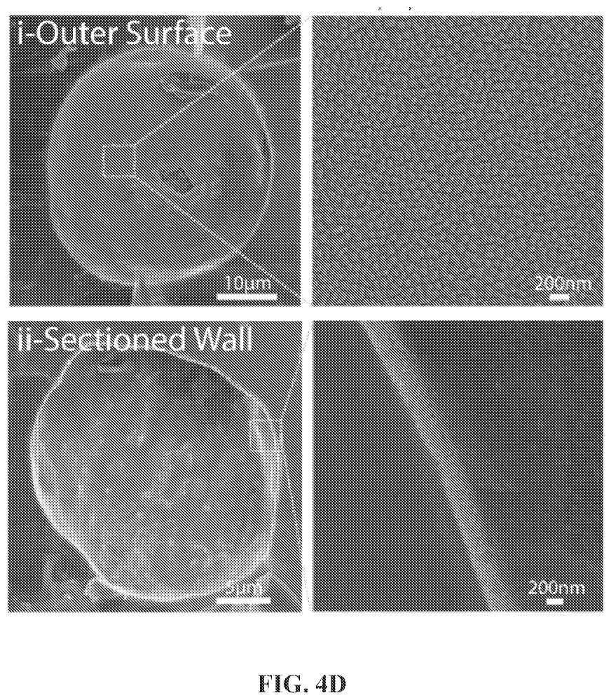

For many downstream applications, POP microparticles require extraction back into an aqueous environment from the emulsion. In addition to stabilizing the microstructures that are formed within water-in-oil emulsions, UV crosslinking through UAA incorporation allows extraction of these microstructures into an all aqueous phase (FIG. 4A). Using a simple de-emulsification process, porous POP microparticles were successfully recovered into buffered saline (FIG. 4B and FIG. 4C). The particles are mechanically stable enough to retain their shape and porosity, albeit with about a 40% reduction in their size following extraction (40.7 μm±1.8 μm pre-extraction, and 25.3 μm±3.4 μm post extraction, n=50 particles each).

To determine their mechanical stability, we used high frequency atomic force microscopy (AFM) to evaluate the Young's modulus (E) of extracted POP microparticles and controls—planar bulk gels of crosslinked ELP (xELP(V)) and a crosslinked POP with 25% helical content (xPOP(V)−25%). While the planar xELP(V) has an E of 0.12±0.02 kPa, the additional stabilization conferred by physical crosslinking increases E of the bulk xPOP(V)−25% by almost one order of magnitude to 1.4±0.3 kPa. Notably, the microparticles of the xPOP(V)−25% were more than an order of magnitude stiffer than the bulk material, with xPOP(V)−25% particles reaching a Young's modulus of 20.9±2.8 kPa, indicating that the ˜40% compaction in the size of the microparticles that occurs post-extraction has an unexpectedly significant effect of its mechanical stiffness. However, the degree of helix incorporation did not have a statistically significant effect on the Young's modulus of crosslinked microparticles, as the Young's moduli of xPOP(V)−25% and xPOP(V)−12.5% were not statistically different, suggesting that any additional physical stabilization conferred by the increased helical content is overwhelmed by the effect of chemical crosslinking and their physical compaction upon extraction from the water-in-oil emulsion.

Crosslinked hollow shells and hollow shell networks can be extracted using the same method as for POP only microparticles (FIG. 4B and FIG. 4D), Despite their thin walls, they are sufficiently strong to tolerate the extraction procedure, though they require hydration to maintain their spherical shape. Imaging the hollow POP shells by cryo-SEM reveals their morphologically rich multiscale architecture. Their remarkably thin walls (FIG. 4D), which range in thickness from approximately 200-400 nm, are composed of tightly packed nanoscale coacervates of POP. These coacervates are architecturally similar to those that make up the bulk POP networks and microparticles. Perhaps due to the influence of the templating ELP core-component, however, they coarsen on a more rapid time scale than the POP microporous networks, which may allow tighter formation of smaller coacervates into a thin, interconnected layer. This packing—which looks similar to tire threads at the outer surface (FIG. 4D)—is the source of the shells' porosity. ELP coacervate that originally forms the core of the shells are, once cooled below their T_{cp} and solubilized, able to traverse the interconnected pathways between the interconnected POP coacervates and diffuse into the surrounding aqueous phase.

Using a set of recombinant IDPs that were designed to exhibit LOST phase behavior, we have created unique microarchitectures in bulk and within microparticles by combining simple, scalable processing techniques with temperature-responsive phase behavior. All of the disclosed architectures are novel in protein-based microparticles and some, such as the ‘fruits-on-a-vine’ and hollow microshell networks, are believed to be unique across classes of materials. These microparticles can be further chemically crosslinked by the incorporation of a genetically encoded UV crosslinkable UAA and brief UV-irradiation. After crosslinking, the covalently stabilized microparticles can be extracted from the water-in-oil emulsion into an all aqueous environment, to provide stable, non-aggregating microparticles. The present disclosure demonstrates that rationally designed artificial IDPs with tunable and programmed aqueous demixing phase behavior can be readily combined with conventional microfluidic polymer processing technology to create novel materials with potentially diverse applications.

It is understood that the foregoing detailed description and accompanying examples are merely illustrative and are not to be taken as limitations upon the scope of the invention. Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A composition comprising: a disordered polypeptide having a transition temperature (Tₜ) and comprising an amino acid sequence of [VPGX¹G]ₘ (SEQ ID NO: 1), wherein X¹ is any amino acid except proline and m is 10 to 500; and a partially ordered polypeptide (POP) having a transition temperature of heating (T_{t-heating}) and a transition temperature of cooling (T_{t-cooling}), and comprising a plurality of disordered domains, wherein each disordered domain includes an amino acid sequence of [VPGX²G]ₙ (SEQ ID NO:12), wherein X² is any amino acid except proline and n is 1 to 200, and a plurality of structured domains, wherein each structured domain includes a polyalanine domain, the polyalanine domain comprising at least 5 alanine residues and having at least about 50% of the amino acids in an alpha-helical conformation, wherein the disordered polypeptide's Tₜ is at least ±1° C. compared to the POP's T_{t-heating}.

Clause 2. A composition comprising an assembly of polypeptides, wherein the polypeptides comprise: a disordered polypeptide having a transition temperature (Tₜ) and comprising an amino acid sequence of [VPGX¹G]ₘ (SEQ ID NO: 1), wherein X¹ is any amino acid except proline and m is 10 to 500; and a partially ordered polypeptide (POP) having a transition temperature of heating (T_{t-heating}) and a transition temperature of cooling (T_{t-cooling}), and comprising a plurality of disordered domains, wherein each disordered domain includes an amino acid sequence of [VPGX²G]ₙ (SEQ ID NO:12), wherein X² is any amino acid except proline and n is 1 to 200, and a plurality of structured domains, wherein each structured domain includes a polyalanine domain, the polyalanine domain comprising at least 5 alanine residues and having at least about 50% of the amino acids in an alpha-helical conformation, wherein the disordered polypeptide's Tₜ is at least ±1° C. compared to the POP's T_{t-heating}.

Clause 3. The composition of clause 2, wherein the assembly comprises: a first assembly including one of the disordered polypeptide and the POP; and a second assembly including the other of the disordered polypeptide and the POP, wherein at least a portion of the first assembly contacts at least a portion of the second assembly.

Clause 4. The composition of anyone of clauses 1-3, wherein the Tₜ is about 10° C. to about 70° C.

Clause 5, The composition of any one of clauses 1-4, wherein the T_{t-heating} and the T_{t-cooling} are both individually about 10° C. to about 70° C.

Clause 6. The composition of any one of clauses 1-5, wherein the T_{t-heating} is at least ±5° C. compared to T_{t-cooling}.

Clause 7. The composition of any one of clauses 1-6, wherein X¹ and X² are both individually Val, Ala, or a combination of Val and Ala.

Clause 8. The composition of any one of clauses 1-7, wherein m is 20 to 200.

Clause 9. The composition of any one of clauses 1-8, wherein n is 5 to 50.

Clause 10, The composition of any one of clauses 1-9, wherein the structured domain comprises an amino acid sequence of (A)₂₅ (SEQ ID NO:15).

Clause 11, The composition of any one of clauses 1-10, wherein the structured domain is present at about 4% to about 75% of the POP based on total number of amino acids.

Clause 12, The composition of anyone of clauses 1-11, wherein the disordered polypeptide, the POP, or both comprise a crosslinking moiety.

Clause 13. The composition of clause 12, wherein the crosslinking moiety comprises a UV crosslinkable amino acid derivative.

Clause 14. The composition of any one of clauses 2-13, wherein the polypeptides self-assemble into the assembly in two phases relative to the T₁ of the disordered polypeptide and the T_{t-heating} of the POP, wherein the two phases comprise: (1) a first phase at a temperature between the Tₜ of the disordered polypeptide and the T_{t-heating} of the POP, wherein one of the disordered polypeptide and the POP self-assembles into a first assembly; and (2) a second phase at a temperature above the Tₜ of the disordered polypeptide and the T_{t-heating} of the POP, wherein the other of the disordered polypeptide and the POP self-assembles into a second assembly, and wherein at least a portion of the second assembly contacts at least a portion of the first assembly.

Clause 15. The composition of any one of clauses 2-14, wherein the assembly comprises: a porous network including the POP, and a plurality of particles contacting the network, each particle including the disordered polypeptide.

Clause 16. The composition of any one of clauses 2-14, wherein the assembly comprises a plurality of particles, each particle comprising: a core including the disordered polypeptide; and a shell including the POP, wherein the shell is positioned on a surface of the core.

Clause 17. A particle comprising the composition of any one of clauses 2-11, wherein the assembly comprises: a porous network including the POP; and a plurality of particles contacting the network, each particle including the disordered polypeptide.

Clause 18. A particle comprising the composition of any one of clauses 2-14, wherein the assembly comprises a network of network particles, each network particle comprising: a core including the disordered polypeptide, and a shell including the POP, wherein the shell is positioned on a surface of the core.

Clause 19. The composition of clause 14, wherein the self-assembly further includes a third phase, the third phase comprising: (3) a third phase at a temperature below the Tₜ of the disordered polypeptide and above the T_{t-cooling} of the POP, wherein the disordered polypeptide dissolves from the assembly.

Clause 20. The composition of clause 19, wherein the assembly comprises: a porous network including the POP; or a plurality of hollow shell particles, each hollow shell particle including the POP.

Clause 21. A particle comprising the composition of clause 19, wherein the assembly comprises: a porous network including the POP; or a network of hollow shell particles, each hollow shell particle including the POP.

Clause 22. The composition or particle of any one of clauses 2-21, wherein the assembly includes crosslinks between the polypeptides.

Clause 23. The composition or particle of any one of clauses 2-22, further comprising a drug molecule encapsulated in the assembly, the first assembly, or the second assembly.

Clause 24. The composition of clause 23, wherein the drug molecule comprises a small molecule, a polypeptide, a polynucleotide, a lipid, a carbohydrate, or a combination thereof.

Clause 25. A cellular scaffold comprising: the composition or particle of any one of clauses 2-24; and a plurality of cells, a drug molecule, or a combination thereof.

Clause 26. A composition comprising a particle of polypeptides, wherein each polypeptide comprises: a partially ordered polypeptide (POP) having a transition temperature of heating (T_{t-heating}) and a transition temperature of cooling (T_{t-cooling}), and comprising a plurality of disordered domains, wherein each disordered domain includes an amino acid sequence of [VPGX²G]ₙ (SEQ ID NO:12), wherein X² is any amino acid except proline and n is 1 to 200, and a plurality of structured domains, wherein each structured domain includes a polyalanine domain, the polyalanine domain comprising at least 5 alanine residues and having at least about 50% of the amino acids in an alpha-helical conformation.

Clause 27. A method of making polypeptide-based assemblies, the method comprising: adding the composition of clause 1 to a first solvent to provide a mixture; heating the mixture to a first temperature between the Tₜ of the disordered polypeptide and the T_{t-heating} of the POP to provide a first assembly including one of the disordered polypeptide and the POP; and heating the mixture to a second temperature above the Tₜ of the disordered polypeptide and the T_{t-heating} of the POP to provide a second assembly including the other of the disordered polypeptide and the POP, wherein at least a portion of the second assembly contacts at least a portion of the first assembly to provide an assembly.

Clause 28. The method of clause 27, further comprising: cooling the mixture to a third temperature below the Tₜ of the disordered polypeptide and above the of the POP, wherein the disordered polypeptide dissolves from the assembly.

Clause 29. The method of clause 27 or clause 28, further comprising emulsifying the mixture prior to heating.

Clause 30. The method of clause 29, wherein the emulsified mixture comprises emulsion droplets, each emulsion droplet including the composition.

Clause 31. The method of clause 30, wherein the first assembly and the second assembly are present within an emulsion droplet.

Clause 32. The method of clause 29, wherein emulsifying the mixture is performed via microfluidics.

Clause 33. The method of any one of clauses 27-32, further comprising exposing the first assembly, the second assembly, or the assembly to a crosslinking agent.

Clause 34. The method of any one of clauses 29-33, further comprising extracting the first assembly, the second assembly, or the assembly into an aqueous phase.

Clause 35. A method of treating a disease in a subject in need thereof, the method comprising administering the composition or particle of any one of clauses 1-24 to the subject.

Sequences
(SEQ ID NO: 1)
(VPGX¹G)ₘ
(SEQ ID NO: 2)
(GVGVP)₈₀
(SEQ ID NO: 3)
(G[V₄/A₁]GVP)₈₀
(SEQ ID NO: 4)
(G[V₁/A₁]GVP)₈₀
(SEQ ID NO: 5)
(G[V₁/A₄]GVP)₈₀
(SEQ ID NO: 6)
((GVGVP)₁₅-GD(A)₂₅K)₄
(SEQ ID NO: 7)
((GVGVP)₃₅-GD(A)₂₅K)₂
(SEQ ID NO: 8)
((G[V₄/A₁]GVP)₁₅-GD(A)₂₅K)₄
(SEQ ID NO: 9)
((G[V₁/A₁]GVP)₁₅-GD(A)₂₅K)₄
(SEQ ID NO: 10)
((G[V₁/A₄]GVP)₁₅-GD(A)₂₅K)₄
(SEQ ID NO: 11)
((G[V₁/A₄]GVP)₃₅-GD(A)₂₅K)₂
(SEQ ID NO: 12)
(VPGX²G)ₙ
(SEQ ID NO: 13)
[Bₚ(A)_{q}Zᵣ]ₙ
(SEQ ID NO: 14)
[(BAₛ)ₜZᵣ]ₙ
(SEQ ID NO: 15)
(A)₂₅
(SEQ ID NO: 16)
K(A)₂₅K
(SEQ ID NO: 17)
(KAAAA)₂₅K
(SEQ ID NO: 18)
GD(A)₂₅K
(SEQ ID NO: 19)
((GVGVP)₁₀-G-A_{z}-GVP(GVGVP)₁₀)₄
(SEQ ID NO: 20)
((GVGVP)₅-G-A_{z}-GVP(GVGVP)₁₀GD(A)₂₅K)₄
(SEQ ID NO: 21)
((G[V₁/A₄]GVP)₅-G-A_{z}-GVP(G[V₁/A₄]GVP)₁₀GD(A)₂₅K)₄
(SEQ ID NO: 22)
((G[V₁/A₄]GVP)₁₀-G-A_{z}-GVP(G[V₁/A₄]GVP)₁₅-G-
A_{z}-GVP(G[V₁/A₄]GVP)₁₀GD(A)₂ₛK)₂
(SEQ ID NO: 23)
MG-(VPGVG)₈₀-GWP
(SEQ ID NO: 24)
MG-[(VPGVG)₃₅-D(A)₂₅K]₂-GWP
(SEQ ID NO: 25)
MG-[(VPGVG)₁₅-D(A)₂₅K]₄-GWP

1. A composition comprising:
a disordered polypeptide having a transition temperature (Tₜ) and comprising an amino acid sequence of [VPGX¹G]ₘ (SEQ ID NO: 1), wherein X¹ is any amino acid except proline and m is 10 to 500; and
a partially ordered polypeptide (POP) having a transition temperature of heating (T_{t-heating}) and a transition temperature of cooling (T_{t-cooling}), and comprising
a plurality of disordered domains, wherein each disordered domain includes an amino acid sequence of [VPGX²G]ₙ (SEQ ID NO:12), wherein X² is any amino acid except proline and n is 1 to 200, and
a plurality of structured domains, wherein each structured domain includes a polyalanine domain, the polyalanine domain comprising at least 5 alanine residues and having at least about 50% of the amino acids in an alpha-helical conformation,
wherein the disordered polypeptide's Tₜ is at least ±1° C. compared to the POP's T_{t-heating}.

2. A composition comprising an assembly of polypeptides, wherein the polypeptides comprise:
a disordered polypeptide having a transition temperature (Tₜ) and comprising an amino acid sequence of [VPGX¹G]ₘ (SEQ ID NO: 1), wherein X¹ is any amino acid except proline and m is 10 to 500; and
a partially ordered polypeptide (POP) having a transition temperature of heating (T_{t-heating}) and a transition temperature of cooling (T_{t-cooling}), and comprising
a plurality of disordered domains, wherein each disordered domain includes an amino acid sequence of [VPGX²G]ₙ (SEQ ID NO:12), wherein X² is any amino acid except proline and n is 1 to 200, and
a plurality of structured domains, wherein each structured domain includes a polyalanine domain, the polyalanine domain comprising at least 5 alanine residues and having at least about 50% of the amino acids in an alpha-helical conformation,
wherein the disordered polypeptide's Tₜ is at least ±1° C. compared to the POP's T_{t-heating}.

3. The composition of claim 2, wherein the assembly comprises:
a first assembly including one of the disordered polypeptide and the POP; and
a second assembly including the other of the disordered polypeptide and the POP,
wherein at least a portion of the first assembly contacts at least a portion of the second assembly.

4. The composition of claim 2, wherein the Tₜ is about 10° C. to about 70° C.

5. The composition of claim 2, wherein the T_{t-heating} and the T_{t-cooling} are both individually about 10° C. to about 70° C.

6. The composition of claim 2, wherein the T_{t-heating} is at least ±5° C. compared to the T_{t-cooling}.

7. The composition of claim 2, wherein X¹ and X² are both individually Val, Ala, or a combination of Val and Ala.

8. The composition of claim 2, wherein m is 20 to 200.

9. The composition of claim 2, wherein n is 5 to 50.

10. The composition of claim 2, wherein the structured domain comprises an amino acid sequence of (A)₂₅ (SEQ ID NO:15).

11. The composition of claim 2, wherein the structured domain is present at about 4% to about 75% of the POP based on total number of amino acids.

12. The composition of claim 2, wherein the disordered polypeptide, the POP, or both comprise a crosslinking moiety.

13. The composition of claim 12, wherein the crosslinking moiety comprises a UV crosslinkable amino acid derivative.

14. The composition of claim 2, wherein the polypeptides self-assemble into the assembly in two phases relative to the Tₜ of the disordered polypeptide and the T_{t-heating} of the POP, wherein the two phases comprise:
(1) a first phase at a temperature between the Tₜ of the disordered polypeptide and the T_{t-heating} of the POP, wherein one of the disordered polypeptide and the POP self-assembles into a first assembly; and
(2) a second phase at a temperature above the Tₜ of the disordered polypeptide and the T_{t-heating} of the POP, wherein the other of the disordered polypeptide and the POP self-assembles into a second assembly, and wherein at least a portion of the second assembly contacts at least a portion of the first assembly.

15. The composition of claim 2, wherein the assembly comprises:
a porous network including the POP; and
a plurality of particles contacting the network, each particle including the disordered polypeptide.

16. The composition of claim 2, wherein the assembly comprises a plurality of particles, each particle comprising:
a core including the disordered polypeptide; and
a shell including the POP, wherein the shell is positioned on a surface of the core.

17. A particle comprising the composition of claim 2, wherein the assembly comprises:
a porous network including the POP; and
a plurality of particles contacting the network, each particle including the disordered polypeptide.

18. A particle comprising the composition of claim 2, wherein the assembly comprises a network of network particles, each network particle comprising:
a core including the disordered polypeptide, and
a shell including the POP, wherein the shell is positioned on a surface of the core.

19. The composition of claim 14, wherein the self-assembly further includes a third phase, the third phase comprising:
(3) a third phase at a temperature below the Tₜ of the disordered polypeptide and above the T_{t-cooling} of the POP, wherein the disordered polypeptide dissolves from the assembly.

20. The composition of claim 19, wherein the assembly comprises:
(i) a porous network including the POP; or
(ii) a plurality of hollow shell particles, each hollow shell particle including the POP.

21. A particle comprising the composition of claim 19, wherein the assembly comprises:
(i) a porous network including the POP; or
(ii) a network of hollow shell particles, each hollow shell particle including the POP.

22. The composition of claim 2, wherein the assembly includes crosslinks between the polypeptides.

23. The composition of claim 2, further comprising a drug molecule encapsulated in the assembly, the first assembly, or the second assembly.

24. The composition of claim 23, wherein the drug molecule comprises a small molecule, a polypeptide, a polynucleotide, a lipid, a carbohydrate, or a combination thereof.

25. A cellular scaffold comprising:
the composition of claim 2; and
a plurality of cells, a drug molecule, or a combination thereof.

26. A composition comprising a particle of polypeptides, wherein each polypeptide comprises:
a partially ordered polypeptide (POP) having a transition temperature of heating (T_{t-heating}) and a transition temperature of cooling (T_{t-cooling}), and comprising
a plurality of disordered domains, wherein each disordered domain includes an amino acid sequence of [VPGX²G]ₙ (SEQ ID NO:12), wherein X² is any amino acid except proline and n is 1 to 200, and
a plurality of structured domains, wherein each structured domain includes a polyalanine domain, the polyalanine domain comprising at least 5 alanine residues and having at least about 50% of the amino acids in an alpha-helical conformation.

27. A method of making polypeptide-based assemblies, the method comprising:
adding the composition of claim 1 to a first solvent to provide a mixture;
heating the mixture to a first temperature between the Tₜ of the disordered polypeptide and the T_{t-heating} of the POP to provide a first assembly including one of the disordered polypeptide and the POP; and
heating the mixture to a second temperature above the Tₜ of the disordered polypeptide and the T_{t-heating} of the POP to provide a second assembly including the other of the disordered polypeptide and the POP, wherein at least a portion of the second assembly contacts at least a portion of the first assembly to provide an assembly.

28.-34. (canceled)

35. A method of treating a disease in a subject in need thereof, the method comprising administering the composition of claim 2 to the subject.
